SEQUENCE LISTING (35 sequences)

SEQ ID NO: 1 - protein, 330 aa, Homo sapiens [sequence omitted]

SEQ ID NO: 2 - protein, 290 aa, Homo sapiens
QMQGVNCTVSSELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVQVHNAKTKPREQQFNSTFRVVSVLTVLHQNWLDGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 3 - protein, 107 aa, Homo sapiens [sequence omitted]

SEQ ID NO: 4 - protein, 11 aa, Mus musculus
GRGDRGDLFGY

SEQ ID NO: 5 - protein, 9 aa, Mus musculus
VNLADAMDY

SEQ ID NO: 6 - protein, 119 aa, Mus musculus
EVKLVESGGGLVKPGGSLKLSCAASGFTFSTYAMSWVRQTPEKRLEWVASISTGDNTYYTDSVRGRFTISRDNARNILYLQMSSLRSEDTAMYFCTRGRGDRGDLFGYWGQGTLVTVSS

SEQ ID NO: 7 - protein, 114 aa, Mus musculus
DIVMSQSPSSLAVSVGEKVTMSCKSSQSLLYRGNQMNYLAWYQQKPGQSPKLLIYWASTRESGVPDRFTGSGSGTEFTLTISSVKAEDLTVYYCQQYYTYPRTFGGGTKLEIKR

SEQ ID NO: 8 - protein, 117 aa, Mus musculus
QVQLKQSGPGLVRPSQSLSITCTVSGFPLGVFGVHWVRQSPGKGLEWLGVIWKGGNTDYNAAFMSRLRISKDNSKSQVFFRMNSLQTDDTAIYYCAKVNLADAMDYWGQGTSVIVSS

SEQ ID NO: 9 - protein, 108 aa, Mus musculus
DIQMTQSPASLSASVGETVTITCRSSGNIHGYLAWFQQKQGKSPQLLVYNTKALAEGVPSRFSGSGSGTQFSLKINNLQPEDFGIYYCQHHYDLPRTFGGGTKLEIKR

SEQ ID NO: 10 - protein, 117 aa, Mus musculus
QVQLKQSGPGLVRPSQSLSITCTVSGFPLGIFGVHWVRQSPGKGLEWLGVIWKGGNTDYNAAFMSRLRITKDNSKSQVFFRMNSLQTDDTAIYYCAKVNLADAMDYWGQGTSVIVSS

SEQ ID NO: 11 - protein, 108 aa, Mus musculus
DIQMTQSPASLSASVGETVTITCRASGNIHGYLAWFQQKQGKSPQLLVYNTKTLAEGVPSRFSGSGSGTQFSLKINSLQPEDFGNYYCQHHYDLPRTFGGGTKLEIKR

SEQ ID NO: 12 - protein, 117 aa, Mus musculus; identical to SEQ ID NO: 10

SEQ ID NO: 13 - protein, 108 aa, Mus musculus; identical to SEQ ID NO: 11

SEQ ID NO: 14 - protein, 117 aa, Artificial
  Generic sequence, with variables
QVQLXXSGPGLVXPSQSLSITCTVSGFPLGAFGVHWVRQSPGKGXEWLGVIWKGGNTDYNAAFXSRLRITKDNSKSQVFFRMNSLQTDDTAXYYCAKVNLADAMDYWGQGTXVXVSS

SEQ ID NO: 15 - protein, 107 aa, Artificial
  Generic sequence, including variables
DIQMTQSPASLSASVGETVTITCRASGNXHGYLAWXQQKXGKXPXLLXYNTKTLAEGVPSRFSGSGSGTXFXXXIXSXQPEDFXXYYCQHHYDLPRTFGGGTKXEIK

SEQ ID NO: 16 - protein, 117 aa, Artificial
  Synthesized sequence
QVQLQESGPGLVKPSQSLSITCTVSGFPLGAFGVHWVRQSPGKGPEWLGVIWKGGNTDYNAAFKSRLRITKDNSKSQVFFRMNSLQTDDTATYYCAKVNLADAMDYWGQGTTVTVSS

SEQ ID NO: 17 - protein, 117 aa, Artificial
  Synthesized sequence
QVQLQESGPGLVKPSQSLSITCTVSGFPLGAFGVHWVRQSPGKGLEWLGVIWKGGNTDYNAAFKSRLRITKDNSKSQVFFRMNSLQTDDTATYYCAKVNLADAMDYWGQGTTVTVSS

SEQ ID NO: 18 - protein, 117 aa, Artificial
  Synthesized sequence
QVQLQESGPGLVKPSQSLSITCTVSGFPLGAFGVHWVRQSPGKGLEWLGVIWKGGNTDYNAAFKSRLRITKDNSKSQVFFRMNSLQTDDTAIYYCAKVNLADAMDYWGQGTTVTVSS

SEQ ID NO: 19 - protein, 107 aa, Artificial
  Synthesized sequence
DIQMTQSPASLSASVGETVTITCRASGNAHGYLAWYQQKPGKAPKLLIYNTKTLAEGVPSRFSGSGSGTQFSLKINSAQPEDFGNYYCQHHYDLPRTFGGGTKVEIK

SEQ ID NO: 20 - protein, 107 aa, Artificial
  Synthesized sequence
DIQMTQSPASLSASVGETVTITCRASGNAHGYLAWYQQKPGKAPKLLVYNTKTLAEGVPSRFSGSGSGTQFSLKINSAQPEDFGNYYCQHHYDLPRTFGGGTKVEIK

SEQ ID NO: 21 - protein, 5 aa, Mus musculus
TYAMS

SEQ ID NO: 22 - protein, 5 aa, Mus musculus
VFGVH

SEQ ID NO: 23 - protein, 5 aa, Mus musculus
IFGVH

SEQ ID NO: 24 - protein, 5 aa, Mus musculus
TFGVH

SEQ ID NO: 25 - protein, 16 aa, Mus musculus
SISTGDNTYYTDSVRG

SEQ ID NO: 26 - protein, 16 aa, Mus musculus
VIWKGGNTDYNAAFMS

SEQ ID NO: 27 - protein, 16 aa, Mus musculus
VIWRGGNTDYNAAFMS

SEQ ID NO: 28 - protein, 17 aa, Mus musculus
KSSQSLLYRGNQMNYLA

SEQ ID NO: 29 - protein, 11 aa, Mus musculus
RSSGNIHGYLA

SEQ ID NO: 30 - protein, 11 aa, Mus musculus
RASGNIHGYLA

SEQ ID NO: 31 - protein, 7 aa, Mus musculus
WASTRES

SEQ ID NO: 32 - protein, 7 aa, Mus musculus
NTKALAE

SEQ ID NO: 33 - protein, 7 aa, Mus musculus
NTKTLAE

SEQ ID NO: 34 - protein, 9 aa, Mus musculus
QQYYTYPRT

SEQ ID NO: 35 - protein, 9 aa, Mus musculus
QHHYDLPRT

(19) United States
(12) Patent Application Publication
(10) Pub. No.: US 2009/0226434 A1
(43) Pub. Date: Sep. 10, 2009

(54) Afucosylated antibodies against CCR5 and their use for the treatment of inflammatory conditions

(75) Inventors: Johannes Auer, Schwaigen (DE); Amy Berson, Palo Alto, CA (US); Dominique Christian Borie, Palo Alto, CA (US); Michael Brandt, Iffeldorf (DE); Jose M. Lora, London (GB); Stefan Ries, Penzberg (DE)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 12/321,016

(22) Filed: Jan. 15, 2009

Related U.S. Application Data
(60) Provisional application No. 61/011,348, filed on Jan. 15, 2008.

(51) Int. Cl. A61K 39/395; C07K 16/18; C12N 5/16; A61P 37/06; A61P 37/02

(52) U.S. Cl. 424/133.1; 530/387.3; 435/328

(57) ABSTRACT

An antibody binding to CCR5 and being glycosylated with a sugar chain at Asn297, said antibody being characterized in that the amount of fucose within said sugar chain is 65% or lower has improved properties in anti-inflammatory therapy.

This application claims priority from U.S. Ser. No. 61/011,348, filed Jan. 15, 2008, incorporated herein by reference in full.

The present invention relates to afucosylated antibodies against CCR5, methods for their production, pharmaceutical compositions containing said antibodies, and their use for the treatment of inflammatory conditions, such as acute and chronic transplant rejection.

### BACKGROUND OF THE INVENTION

Chemokines and their receptors are known to participate in allograft rejection by mediating leukocyte trafficking. U. Panzer et al. (Transplantation (2004) 78:1341-50) reported CCR5-positive T cell recruitment in acute human allograft rejection, and B. Luckow et al. (Eur. J. Immunol. (2004) 34:2568-78) observed decreased intragraft levels of metalloproteinases and arteriosclerosis in CCR5-deficient animals. Further, W. Gao et al. (Transplantation (2001) 72:1199-205) demonstrated prolonged allograft survival in mice treated with CCR5-specific monoclonal antibody and in CCR5-deficient mice. C. Schroeder et al., J. Immunol. (2007) 179:2289-99, explored the effects of a CCR5 antagonist in a cynomolgus monkey cardiac allograft model for investigation of CCR5 modulation during inflammation and alloimmunity. Moreover, a retrospective study in human transplant recipient cohorts uncovered that CCR5-deficient patients (delta 32) showed prolonged allograft survival (M. Fischereder et al., Lancet (2001) 357:1758-61).

In experimental models, due to the redundancy of receptor-ligand interaction, the deficiency or blockade of a single chemokine does not protect the allograft from acute rejection (M. Fischereder et al., supra; W. Gao et al., supra; W. W. Hancock et al., Curr. Opin. Immunol. (2000) 12:511; W. W. Hancock et al., Curr. Opin. Immunol. (2003) 15:479).

WO01/78707 refers to a method of inhibiting graft rejection comprising administering an antagonist of CCR5 function.

Cell-mediated effector functions of monoclonal antibodies can be enhanced by engineering their oligosaccharide component as described in P. Umaña et al., Nature Biotechnol. (1999) 17:176-80, and U.S. Pat. No. 6,602,684. IgG1 type antibodies, the most commonly used antibodies in cancer immunotherapy, are glycoproteins that have a conserved N-linked glycosylation site at Asn297 in each C_{H}2 domain. The two complex biantennary oligosaccharides attached to Asn297 are buried between the C_{H}2 domains, forming extensive contacts with the polypeptide backbone, and their presence is essential for the antibody to mediate effector functions such as antibody dependent cellular cytotoxicity (ADCC) (M. R. Lifely et al., Glycobiology (1995) 5:813-22; R. Jefferis et al., Immunol. Rev. (1998) 163:59-76; A. Wright and S. L. Morrison, Trends Biotechnol. (1997) 15:26-32). P. Umaña et al., supra, and WO 99/54342 showed that overexpression in Chinese hamster ovary (CHO) cells of β-(1,4)-N-acetylglucosaminyltransferase III (“GnTIII”), a glycosyltransferase catalyzing the formation of bisected oligosaccharides, significantly increases the in vitro ADCC activity of antibodies. Alterations in the composition of the N297 carbohydrate or its elimination affect also binding to FcγR and C1q (P. Umaña et al., supra; Davies et al., Biotechnol. Bioeng. (2001) 74:288-94; Y. Mimura et al., J. Biol. Chem. (2001) 276:45539-47; Radaev et al., J. Biol. Chem. (2001) 276:16478-83; R. L. Shields et al., J. Biol. Chem. (2001) 276:6591-604; R. L. Shields et al., J. Biol. Chem. (2002) 277:26733-40; L. C. Simmons et al., J. Immunol. Meth (2002) 263:133-47).

S. Iida et al., Clin. Cancer Res. (2006) 12:2879-87, show that efficacy of a nonfucosylated anti-CD20 antibody was inhibited by addition of fucosylated anti-CD20 antibodies. The efficacy of a 1:9 mixture (10 μg/mL) of nonfucosylated and fucosylated anti-CD20 antibodies was inferior to that of a 1,000-fold dilution (0.01 μg/mL) of nonfucosylated anti-CD20 antibody alone. They conclude that nonfucosylated IgG1, not including fucosylated counterparts, can evade the inhibitory effect of plasma IgG on ADCC through its high FcgammaRIIIa binding. A. Natsume et al., show in J. Immunol. Methods (2005) 306:93-103, that fucose removal from complex-type oligosaccharide of human IgG 1-type antibody results in a great enhancement of antibody-dependent cellular cytotoxicity (ADCC). M. Satoh et al., Expert Opin. Biol. Ther. (2006) 6:1161-73, discuss non-fucosylated therapeutic antibodies as next-generation therapeutic antibodies. Satoh concludes that antibodies consisting of only the non-fucosylated human IgG1 form are thought to be ideal. Y. Kanda et al., Biotechnol. Bioeng. (2004) 94:680-88, compared fucosylated anti-CD20 antibody (96% fucosylation, CHO/DG44 1H5) with non-fucosylated anti-CD20 antibody. J. Davies et al., Biotechnol. Bioeng. (2001) 74:288-94, report that for an anti-CD20 antibody increased ADCC correlates with increased binding to FcγRIII.

Methods to enhance cell-mediated effector functions of monoclonal antibodies are described, e.g., in WO 2005/018572, WO 2006/116260, WO 2006/114700, WO 2004/065540, WO 2005/011735, WO 2005/027966, WO 1997/028267, US 2006/0134709, US 2005/0054048, US 2005/0152894, WO 2003/035835, WO 2000/061739.

### SUMMARY OF THE INVENTION

The invention comprises an antibody binding to CCR5, and being glycosylated with a sugar chain at Asn297, said antibody being characterized in that the amount of fucose within said sugar chain is 65% or lower.

The invention comprises an antibody binding to CCR5, and being glycosylated with a sugar chain at Asn297, said antibody being characterized in that the amount of fucose within said sugar chain is preferably between 5% and 65%, and further preferably between 20% and 40%. Antibodies according to the invention comprising such amount of fucose are further termed as afucosylated.

The invention comprises an antibody binding to CCR5, and being glycosylated with a sugar chain at Asn297, said antibody being characterized in showing high binding affinity to the FcγRIII.

In one subgroup of the invention, the antibody is of human IgG1, or IgG3 type.

In one subclass of the invention, the amount of NGNA is 1% or less and/or the amount of N-terminal alpha-1,3-galactose is 1% or less within said sugar chain.

In another subclass of the invention, the amount of NGNA is 0.5% or less, more preferably 0.1% or less and even not detectable (LCMS).

In another subclass of the invention, the amount of N-terminal alpha-1,3-galactose within said sugar chain is 0.5% or less, more preferably 0.1% or less and even not detectable (LCMS).

The sugar chain can show the characteristics of N-linked glycans attached to Asn297 of an antibody binding to CCR5 recombinantly expressed in a CHO cell.

One subclass of the invention comprises the method of inhibiting human CCR5, comprising contacting said CCR5 with an afucosylated antibody that binds to CCR5, characterized in that said antibody binds to human CCR5, and inhibits or blocks its function so that CCR5+ T-cells in vitro and in vivo are depleted. Another subclass of the invention comprises the method of preventing allograft rejection, inflammation, and immune-mediated diseases. In one embodiment of the invention, the disease is rheumatoid arthritis (RA), chronic obstructive pulmonary disease (COPD), or granulomatous colitis and regional enteritis (Crohn's disease).

Antibodies according to the invention show benefits for patients in need of inhibiting graft rejection.

In another subclass of the invention, the antibody is a monoclonal antibody and, in addition, a chimeric antibody (human constant chain), a humanized antibody or a human antibody.

The invention further comprises a pharmaceutical composition containing an antibody according to the invention, optionally together with a buffer and/or an adjuvant useful for the formulation of antibodies for pharmaceutical purposes.

The invention further comprises a pharmaceutical composition comprising an antibody according to the invention.

The invention further provides pharmaceutical compositions comprising an antibody according to the invention and a pharmaceutically acceptable carrier. In one embodiment, the pharmaceutical composition may be included in an article of manufacture or kit. The invention further provides the use of an antibody according to the invention for the manufacture of a pharmaceutical composition for the treatment of graft rejection. The antibody is used in a pharmaceutically effective amount.

The invention further comprises the use of an antibody according to the invention for the manufacture of a pharmaceutical composition for the prevention of allograft rejection and inflammation and immune-mediated diseases. The antibody is used in a pharmaceutically effective amount.

The invention further comprises a method for the production of a recombinant human antibody according to the invention, characterized by expressing a nucleic acid encoding an antibody binding to CCR5 in a CHO host cell, which fucosylates said antibody in an amount according to the invention, and recovering said antibody from said cell. The invention further comprises the antibody obtainable by such a recombinant method.

The invention further comprises a CHO cell capable of recombinantly expressing β(1,4)-N-acetylglucosaminyltransferase III (GnTIII), mannosidase II (ManII), and an anti-CCR5 antibody. Such a CHO cell is a CHO cell, transformed with a first DNA sequence encoding a polypeptide having GnTIII activity and a polypeptide having ManII activity and a polypeptide having ManII activity, a second DNA sequence encoding at least the variable domain of the heavy chain of an antibody against CCR5, and a third DNA sequence encoding at least the variable domain of the light chain of an antibody against CCR5. Preferably the second and third DNA sequences encode the heavy and light chain of an antibody against CCR5 of human IgG1 type.

The invention further comprises a process for the production of an antibody against CCR5 showing the properties according to the invention, comprising the steps of trans-forming a host cell, preferably a CHO cell, with a first DNA sequence encoding a polypeptide having GnTIII activity, a second DNA sequence encoding at least the variable domain of the heavy chain of an antibody against CCR5, and a third DNA sequence encoding at least the variable domain of the light chain of an antibody against CCR5, cultivating in a fermentation medium said host cell, which expresses, preferably independently, said first, second and third DNA sequences, under conditions that said host cell secretes said antibody to the fermentation medium, and isolating said antibody from the fermentation medium.

The present invention further relates to a method of treating or preventing acute and chronic organ transplant rejection (allograft, xenograft) in a mammal, including a human, comprising administering to said mammal an antibody against CCR5 according to the invention.

### DETAILED DESCRIPTION OF THE INVENTION

The term “CCR5” denotes a human chemokine receptor (see e.g. Swiss Prot P51681 and Mueller, A., and Strange, P. G., Int. J. Biochem. Cell Biol. 36 (2004) 35-38). The term “CCR5 antibody” means an antibody against CCR5, an anti-CCR5 antibody.

The term “antibody” encompasses the various forms of antibodies including but not being limited to whole antibodies, antibody fragments, human antibodies, humanized antibodies and genetically engineered antibodies as long as the characteristic properties according to the invention are retained.

“Antibody fragments” comprise a portion of a full length antibody, generally at least the antigen binding portion or the variable region thereof. Examples of antibody fragments include diabodies, single-chain antibody molecules, immunotoxins, and multispecific antibodies formed from antibody fragments.

The terms “monoclonal antibody” or “monoclonal antibody composition” as used herein refer to a preparation of antibody molecules of a single amino acid composition. Accordingly, the term “human monoclonal antibody” refers to antibodies displaying a single binding specificity which have variable and constant regions derived from human germline immunoglobulin sequences.

The term “chimeric antibody” refers to a monoclonal antibody comprising a variable region, i.e. binding region, from one source or species and at least a portion of a constant region derived from a different source or species, usually prepared by recombinant DNA techniques. Chimeric antibodies comprising a murine variable region and a human constant region are especially preferred. Such murine/human chimeric antibodies are the product of expressed immunoglobulin genes comprising DNA segments encoding murine immunoglobulin variable regions and DNA segments encoding human immunoglobulin constant regions. Other forms of “chimeric antibodies” encompassed by the present invention are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. See, e.g., Morrison, S. L., et al., Proc. Natl. Acad. Sci. USA 81 (1984) 6851-6855; U.S. Pat. Nos. 5,202,238 and 5,204,244.

The term “humanized antibody” refers to antibodies in which the framework regions and/or “complementarity determining regions” (CDRs) have been modified to comprise the CDR of an immunoglobulin of different specificity as compared to that of the parent immunoglobulin. In a preferred embodiment, a murine CDR is grafted into the framework region of a human antibody to prepare a “humanized antibody.” See, e.g., Riechmann, L., et al., Nature 332 (1988) 323-327; and Neuberger, M. S., et al., Nature 314 (1985) 268-270. Particularly preferred CDRs correspond to those representing sequences recognizing the antigens noted herein for chimeric and bifunctional antibodies. Preferably said antigens are epitopes of CCR5.

The term “human antibody”, as used herein, is intended to include antibodies having variable and constant regions derived from human germline immunoglobulin sequences. The variable heavy chain region is preferably derived from germline sequence DP-50 (GenBank LO6618) and the variable light chain region is preferably derived from germline sequence L6 (GenBank X01668) or the variable heavy chain region is preferably derived DP-61 (GenBank M99682) and the variable light chain region is derived from germline sequence L15 (GenBank K01323). The constant regions of the antibody are constant regions of human IgG1 type. Such regions can be allotypic and are described by, e.g., Johnson, G., and Wu, T. T., Nucleic Acids Res. 28 (2000) 214-218, and the databases referenced therein.

The term “recombinant human antibody”, refers to antibodies having variable and constant regions derived from human germline immunoglobulin sequences in a rearranged form. The recombinant human antibodies according to the invention have been subjected to in vivo somatic hypermutation. Thus, the amino acid sequences of the variable heavy chain regions (VH) and variable light chain regions (VL) of the recombinant antibodies are sequences that, while been derived from and are related to human germline VH and VL sequences, may not naturally exist within the human antibody germline repertoire in vivo.

As used herein, the term “binding” refers to antibody binding to CCR5 with an affinity of about 10⁻¹³ to 10⁻⁸ M (K_{D}), preferably of about 10⁻¹³ to 10⁻⁹ M.

The term “nucleic acid molecule”, as used herein, is intended to include DNA molecules and RNA molecules. A nucleic acid molecule may be single-stranded or double-stranded, but preferably is double-stranded DNA.

Human constant regions having IgG1 or IgG3 type are described in detail by Kabat et al., Sequences of Proteins of Immunological Interest, 5th Ed. Public Health Service, National Institutes of Health, Bethesda, Md. (1991), and by Brüggemann, M., et al., J. Exp. Med. 166 (1987) 1351-1361; Love, T. W., et al., Methods Enzymol. 178 (1989) 515-527. Examples are shown in SEQ ID NO: 01 to 03.

Constant regions of human IgG1, IgG2 or IgG3 type are glycosylated at Asn297. “Asn297” according to the invention means amino acid asparagine located at about position 297 in the Fc region; based on minor sequence variations of antibodies, Asn297 can also be located some amino acids (usually not more than ±3 amino acids) upstream or downstream of position 297, i.e. between position 294 and 300.

The “variable region” (variable region of a light chain (VL), variable region of a heavy chain (VH)) as used herein denotes each of the pair of light and heavy chain domains which is involved directly in binding of the antibody to the antigen. The variable regions of human light and heavy chains have the same general structure, each comprising four framework (FR) regions, whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody heavy and light chain variable region CDR3 regions play a particularly important role in the binding specificity/affinity of the antibodies according to the invention and therefore provide a further object of the invention.

The terms “hypervariable region” or “antigen-binding portion of an antibody” when used herein refer to the amino acid residues of an antibody which are responsible for antigen-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. Especially, CDR3 of the heavy chain is the region which contributes most to antigen binding and characterizes the antibody. CDR and FR regions are determined according to the standard definition of Kabat et al., Sequences of Proteins of Immunological Interest, 5th Ed. Public Health Service, National Institutes of Health, Bethesda, Md. (1991)) and/or those residues from a “hypervariable loop”.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing agents.

Antibody A (DSM ACC 2683) binds to an epitope including amino acids on the ECL2 domain of CCR5 (Lee, B., et al., J. Biol. Chem. 274 (1999) 9617-9626) which is different from the epitope recognized by antibody 2D7 (2D7 binds to amino acids K171 and E172 of ECL2A but not to ECL2B amino acids 184-189). Epitope binding for antibody A is found to be 20% for CCR5 mutant K171A or E172A (if glu172 is mutated to ala). 100% epitope binding is defined for wild-type CCR5. A further embodiment of the invention is therefore an afucosylated antibody binding to CCR5 and to the same epitope as antibody A binds. Binding inhibition can be detected by an SPR assay using immobilized antibody A and CCR5 at a concentration of 20-50 nM and the antibody to be detected at a concentration of 100 nM. A signal reduction of 50% or more shows that the antibody competes with antibody A. Epitope binding is investigated by using alanine mutation of CCR5 according to the method described by Olson, W. C. et al., J. Virol. 73 (1999) 4145-4155, for epitope mapping. A signal reduction of 75% or more shows that the mutated amino acid(s) contribute to the epitope of said antibody. Binding to the same epitope is found, if the amino acids contributing to the epitope of the investigated antibody and the amino acids contributing to the epitope of antibody A are identical. Preferably the antibody according to the invention binds to the ECL2 domain of CCR5.

Amino acid sequences of preferred CCR5 antibodies according to the invention are described in WO2006/103100 and WO2008/037419.

One aspect of the invention comprises an afucosylated antibody binding to CCR5 characterized in that the variable heavy chain amino acid sequence CDR3 of said antibody is selected from the heavy chain CDR3 sequences SEQ ID NO: 04 or 05.

The antibody can be characterized in containing as heavy chain CDRs the CDRs of SEQ ID NO: 06 and as light chain CDRs the CDRs of SEQ ID NO: 07, as heavy chain CDRs the CDRs of SEQ ID NO: 08 and as light chain CDRs the CDRs of SEQ ID NO: 09, as heavy chain CDRs the CDRs of SEQ ID NO: 10 and as light chain CDRs the CDRs of SEQ ID NO: 11, as heavy chain CDRs the CDRs of SEQ ID NO: 12 and as light chain CDRs the CDRs of SEQ ID NO: 13, as heavy chain CDRs the CDRs of SEQ ID NO: 14 and as light chain CDRs the CDRs of SEQ ID NO: 15, as heavy chain CDRs the CDRs of SEQ ID NO: 16 and as light chain CDRs the CDRs of SEQ ID NO: 19, as heavy chain CDRs the CDRs of SEQ ID NO: 16 and as light chain CDRs the CDRs of SEQ ID NO: 20, as heavy chain CDRs the CDRs of SEQ ID NO: 17 and as light chain CDRs the CDRs of SEQ ID NO: 19, as heavy chain CDRs the CDRs of SEQ ID NO: 17 and as light chain CDRs the CDRs of SEQ ID NO: 20, as heavy chain CDRs the CDRs of SEQ ID NO: 18 and as light chain CDRs the CDRs of SEQ ID NO: 19, or as heavy chain CDRs the CDRs of SEQ ID NO: 18 and as light chain CDRs the CDRs of SEQ ID NO: 20.

Another aspect of the invention comprises an afucosylated antibody binding to CCR5, characterized in that the heavy chain variable domain comprises an amino acid sequence of the formula

(SEQ ID NO: 14)

Gln-Val-Gln-Leu-X01-X02-Ser-Gly-Pro-Gly-Leu-Val-
X03-Pro-Ser-Gln-Ser-Leu-Ser-Ile-Thr-Cys-Thr-Val-
Ser-Gly-Phe-Pro-Leu-Gly-Ala-Phe-Gly-Val-His-Trp-
Val-Arg-Gln-Ser-Pro-Gly-Lys-Gly-X04-Glu-Trp-Leu-
Gly-Val-Ile-Trp-Lys-Gly-Gly-Asn-Thr-Asp-Tyr-Asn-
Ala-Ala-Phe-X05-Ser-Arg-Leu-Arg-Ile-Thr-Lys-Asp-
Asn-Ser-Lys-Ser-Gln-Val-Phe-Phe-Arg-Met-Asn-Ser-
Leu-Gln-Thr-Asp-Asp-Thr-Ala-X06-Tyr-Tyr-Cys-Ala-
Lys-Val-Asn-Leu-Ala-Asp-Ala-Met-Asp-Tyr-Trp-Gly-
Gln-Gly-Thr-X07-Val-X08-Val-Ser-Ser,
wherein
X01 is Lys or Gln,
X02 is Gln or Glu,
X03 is Arg or Lys,
X04 is Leu or Pro,
X05 is Met or Lys,
X06 is Ile or Thr,
X07 is Ser or Thr,
X08 is Ile or Thr.

A subclass of the invention is the antibody is characterized in that the light chain variable domain of said antibody comprises an amino acid sequence of the formula

(SEQ ID NO: 15)

Asp-Ile-Gln-Met-Thr-Gln-Ser-Pro-Ala-Ser-Leu-Ser-
Ala-Ser-Val-Gly-Glu-Thr-Val-Thr-Ile-Thr-Cys-Arg-
Ala-Ser-Gly-Asn-X10-His-Gly-Tyr-Leu-Ala-Trp-X11-
Gln-Gln-Lys-X12-Gly-Lys-X13-Pro-X14-Leu-Leu-X15-
Tyr-Asn-Thr-Lys-Thr-Leu-Ala-Glu-Gly-Val-Pro-Ser-
Arg-Phe-Ser-Gly-Ser-Gly-Ser-Gly-Thr-X16-Phe-X17-
X18-X19-Ile-X20-Ser-X21-Gln-Pro-Glu-Asp-Phe-X22-
X23-Tyr-Tyr-Cys-Gln-His-His-Tyr-Asp-Leu-Pro-Arg-
Thr-Phe-Gly-Gly-Gly-Thr-Lys-X24-Glu-Ile-Lys,
wherein
X10 is Ile or Ala,
X11 is Phe or Tyr,
X12 is Gln or Pro,
X13 is Ser or Ala,
X14 is Gln or Lys,
X15 is Val or Ile,
X16 is Gln or Asp,
X17 is Ser or Thr,
X18 is Leu or Ala,
X19 is Lys or Thr,
X20 is Asn or Ser,
X21 is Leu or Ala,
X22 is Gly or Ala,
X23 is Asn or Thr,
X24 is Leu or Val.

Another aspect of the invention is the antibody that binds to CCR5 and comprises a variable heavy or light chain domain selected from the group of variable domains comprising heavy chain variable domains of SEQ ID NO: 14, light chain variable domains of SEQ ID NO: 15, or a CCR5-binding fragment thereof.

One class of the invention comprises the antibody characterized in that the constant regions (light and heavy chains) are of human origin. Such constant regions (chains) are known in the state of the art, for example as described by Kabat (see, e.g., G. Johnson and T. T. Wu, Nucleic Acids Res. (2000) 28:214-18). For example, a useful human heavy chain constant region comprises an amino acid sequence independently selected from SEQ ID NO: 01 or 02. For example, a useful human light chain constant region comprises an amino acid sequence of a kappa-light chain constant region of SEQ ID NO: 03. In one aspect of the invention, the antibody is of mouse origin and comprises the antibody variable sequence frame of a mouse antibody according to Kabat (see, e.g., Johnson and Wu, supra).

The antibodies inhibit one or more functions of human CCR5, such as ligand binding to CCR5, signaling activity (e.g. activation of a mammalian G protein, induction of a rapid and transient increase in the concentration of cytosolic free Ca²⁺, and/or stimulation of a cellular response (e.g. stimulation of chemotaxis, exocytosis or inflammatory mediator release by leukocytes, integrin activation)). The antibodies inhibit binding of RANTES, MIP-1 alpha, and/or MIP-1 beta, to human CCR5 and/or inhibit functions mediated by human CCR5, like leukocyte trafficking, T cell activation, inflammatory mediator release, and/or leukocyte degranulation.

An antibody according to the invention preferably does not inhibit chemokine binding in a binding assay to CCR1, CCR2, CCR3, CCR4, CCR6, and CXCR4 in an antibody concentration up to 100 μg/ml.

Glycosylation of human IgG1 or IgG3 occurs at Asn297 as core fucosylated biantennary complex oligosaccharide glycosylation terminated with up to two Gal residues. These structures are designated as G0, G1 (α-1,6- or α-1,3-), or G2 glycan residues, depending from the amount of terminal Gal residues (Raju, T. S., BioProcess Int., April 2003, 44-53). CHO type glycosylation of antibody Fc parts is e.g. described by Routier, F. H., Glycoconjugate J. 14 (1997) 201-207. Antibodies which are recombinantly expressed in nonglycomodified CHO host cells usually are fucosylated at Asn297 in an amount of at least 85%.

According to the invention “amount of fucose” means the amount of said sugar within the sugar chain at Asn297, related to the sum of all glycostructures attached to Asn297 (e.g. complex, hybrid and high mannose structures) measured by MALDI-TOF mass spectrometry and calculated as average value (see example 8). The relative amount of fucose is the percentage of fucose-containing structures related to all glycostructures identified in an N-Glycosidase F treated sample (e.g. complex, hybride and oligo- and high-mannose structures, resp.) by MALDI-Tof.

The afucosylated anti-CCR5 antibody according to the invention can be expressed in a glycomodified host cell engineered to express at least one nucleic acid encoding a poly-peptide having GnTIII activity and a polypeptide having ManII activity in an amount to fucosylate according to the invention the oligosaccharides in the Fc region. In one embodiment, the polypeptide having GnTIII activity is a fusion polypeptide. Alternatively α1,6-fucosyltransferase activity of the host cell can be decreased or eliminated according to U.S. Pat. No. 6,946,292 to generate glycomodified host cells. The amount of antibody fucosylation can be predetermined e.g. either by fermentation conditions or by combination of at least two antibodies with different fucosylation amount.

The anti-CCR5 antibody according to the invention can be produced in a host cell by a method comprising: (a) culturing a host cell engineered to express at least one polynucleotide encoding a fusion polypeptide having GnTIII activity and/or ManII activity under conditions which permit the production of said antibody and which fucosylation of the oligosaccharides present on the Fc region of said antibody according to the invention; and (b) isolating said antibody. In one embodiment, the polypeptide having GnTIII activity is a fusion polypeptide, preferably comprising the catalytic domain of GnTIII and the Golgi localization domain of a heterologous Golgi resident polypeptide selected from the group consisting of the localization domain of mannosidase II, the localization domain of β(1,2)-N-acetylglucos-aminyltransferase I (“GnTI”), the localization domain of marmosidase I, the localization domain of β(1,2)-N-acetylglucosaminyltransferase II (“GnTII”), and the localization domain of α1-6 core fucosyltransferase. Preferably, the Golgi localization domain is from mannosidase II or GnTI. In a further aspect, the invention is directed to a method for modifying the glycosylation profile of an anti-CCR5 antibody by using such method.

In another aspect, the present invention is directed to a method of modifying the glycosylation of an anti-CCR5 antibody by using a fusion polypeptide having GnTIII activity and comprising the Golgi localization domain of a heterologous Golgi resident polypeptide. In one embodiment, the fusion polypeptides of the invention comprise the catalytic domain of GnTIII. In another embodiment, the Golgi localization domain is selected from: the localization domain of mannosidase II, the localization domain of GnTI, the localization domain of mannosidase I, the localization domain of GnTII, or the localization domain of α1-6 core fucosyltransferase. Preferably, the Golgi localization domain is from mannosidase II or GnTI.

According to the present invention, these modified oligosaccharides of the anti-CCR5 antibody may be hybrid or complex. Preferably the bisected, nonfucosylated oligo-saccharides are hybrid. In another embodiment, the bisected, nonfucosylated oligosaccharides are complex.

As used herein, a “polypeptide having GnTIII activity” refers to polypeptides that are able to catalyze the addition of an N-acetylglucosamine (GlcNAc) residue in β-1-4 linkage to the β-linked mannoside of the trimannosyl core of N-linked oligosaccharides. This includes fusion polypeptides exhibiting enzymatic activity similar to, but not necessarily identical to, an activity of β-(1,4)-N-acetylglucosaminyltransferase III, also known as β-1,4-mannosyl-glycoprotein 4-beta-N-acetylglucosaminyl-transferase (EC 2.4.1.144), according to the Nomenclature Committee of the International Union of Biochemistry and Molecular Biology (NC-IUBMB), as measured in a particular biological assay, with or without dose dependency. In the case where dose dependency does exist, it need not be identical to that of GnTIII, but rather substantially similar to the dose-dependence in a given activity as compared to the GnTIII (i.e. the candidate polypeptide will exhibit greater activity or not more than about 25-fold less and, preferably, not more than about tenfold less activity, and most preferably, not more than about three-fold less activity relative to the GnTIII). As used herein, the term “Golgi localization domain” refers to the amino acid sequence of a Golgi resident polypeptide which is responsible for anchoring the polypeptide in location within the Golgi complex. Generally, localization domains comprise amino terminal “tails” of an enzyme.

The antibodies according to the invention show high binding affinity to the Fc gamma receptor III (FcγRIII, CD16a). High binding affinity to FcγRIII denotes that binding is enhanced for CD16a/F158 at least 10-fold in relation to the wildtype anti-CCR5 antibody (95% fucosylation) as standard (see example 5) expressed in CHO host cells, such as CHO DG44 or CHO K1 cells, and binding is enhanced for CD16a/V158 at least 20-fold in relation to the wildtype anti-CCR5 antibody measured by Surface Plasmon Resonance (SPR) using immobilized CD16a at an antibody concentration of 100 nM (see example 3). FcγRIII binding can be increased by methods according to the state of the art by modifying the amino acid sequence of the Fc part or the glycosylation of the Fc part of the antibody. Preferred methods are described above.

The term “binding to CCR5” as used herein means the binding of the antibody to CCR5 in an in vitro assay, preferably in a binding assay in which the antibody is bound to a surface and binding of CCR5 is measured by Surface Plasmon Resonance (SPR). Binding means a binding affinity (K_{D}) of 10⁻⁸ M or less, preferably 10⁻¹³ M to 10⁻⁹ M.

Binding of the antibody to CCR5 or FcγRIII can be investigated by a BIAcore assay (Pharmacia Biosensor AB, Uppsala, Sweden). The affinity of the binding is defined by the terms ka (rate constant for the association of the antibody from the antibody/antigen complex), kd (dissociation constant), and K_{D} (kd/ka). The antibodies according to the invention show a K_{D} of 10⁻⁸ M or less for the binding to CCR5.

The term “CCR5 expressing cells” refers to such cells which are
a) naturally expressing CCR5 (such as CD4+ and CD8+ T-cells, as well as monocytes and other immune cells),
b) recombinant, engineered mouse L1.2 cells (ATCC HB204) and CHO cells (CHO K1—ATCC CCL-61, CHO DG44—Urlaub et al. Cell 33 (1983) 405-412), or other cell lines
c) cells expressing CCR5 after stimulation with cytokines, HIV, sodium butyrate or other stimuli.

The term “CCR5+” denotes cell expressing and presenting the chemokine receptor CCR5 on it outer cell membrane surface.

The term “antibody-dependent cellular cytotoxicity (ADCC)” refers to lysis of human target cells by an antibody according to the invention in the presence of effector cells. ADCC is measured preferably by the treatment of a preparation of CCR5 expressing cells with an antibody according to the invention in the presence of effector cells such as freshly isolated PBMC or purified effector cells from buffy coats, like monocytes or natural killer (NK) cells or a permanently growing NK cell line.

As used herein, the term host cell covers any kind of cellular system which can be engineered to generate the polypeptides and antigen-binding molecules of the present invention. In one embodiment, the host cell is able and engineered to allow the production of an antigen binding molecule with modified glycoforms. The host cells have been further manipulated to express increased levels of one or more polypeptides having GnTIII activity. CHO cells are preferred as host cells.

For the protein expression in the host cell, nucleic acids encoding light and heavy chains or fragments thereof are inserted into expression vectors by standard methods. Expression is performed in such host cells, and the antibody is recovered from the cells (supernatant or cells after lysis).

The general methods for recombinant production of antibodies are well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-161; Werner, R. G., Drug Res. 48 (1998) 870-880.

The antibodies may be present in whole cells, in a cell lysate, or in a purified form. Purification is performed in order to eliminate other cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al., ed. Current Protocols in Molecular Biology, Greene Publishing and Wiley Interscience, New York (1987).

The control sequences that are suitable for prokaryotes, for example, include a promoter, optionally an operator sequence, and a ribosome binding site. Eukaryotic cells are known to utilize promoters, enhancers and polyadenylation signals.

A nucleic acid is “operably linked” when it is placed in a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The monoclonal antibodies are suitably separated from the culture medium by conventional immunoglobulin purification procedures such as, for example, protein A-Sepharose chromatography, hydroxylapatite chromatography, gel electrophoresis, dialysis, or affinity chromatography. DNA and RNA encoding the monoclonal antibody is readily isolated and sequenced using conventional procedures. The hybridoma cells can serve as a source of such DNA and RNA.

One embodiment of the invention is a method for treating a patient susceptible to allograft rejection, and for the treatment of inflammation and other immune-mediated diseases, comprising administering to the patient an effective amount of an antibody according to the invention.

Another embodiment of the invention is a method for the treatment of a patient suffering from graft rejection or graft versus host disease, characterized by administering to the patient an antibody according to the invention.

Another aspect of the invention is a pharmaceutical composition comprising an effective amount of an antibody according to the invention, and a pharmaceutically acceptable carrier.

Another aspect of the invention is a pharmaceutical composition comprising an effective amount of an antibody according to the invention, a calcineurin inhibitor, and a pharmaceutically acceptable carrier.

Another embodiment of the invention is a method for the manufacture of a pharmaceutical composition comprising an antibody according to the invention.

In another aspect, the present invention provides a pharmaceutical composition, containing an antibody of the present invention, formulated together with a pharmaceutical carrier.

As used herein, “pharmaceutical carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g. by injection or infusion).

A composition of the present invention can be administered by a variety of methods known in the art. As will be appreciated by the skilled artisan, the route and/or mode of administration will vary depending upon the desired results.

To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions.

Pharmaceutical carriers include sterile aqueous solutions or dispersions and sterile powders for the extemporaneous preparation of sterile injectable solutions or dispersion. The use of such media and agents for pharmaceutically active substances is known in the art.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The composition must be sterile and fluid to the extent that the composition is deliverable by syringe. In addition to water, the carrier preferably is an isotonic buffered saline solution.

Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition.

Transplantation is performed according to the state of the art with numerous cell types, tissue types and organ types, e.g. pancreatic islets, corneal, bone marrow, stem cells, skin graft, skeletal muscle, aortic and aortic valves, and organs as heart, lung, kidney, liver, and pancreas.

The invention comprises the use of the antibodies according to the invention for the treatment of a patient suffering from GvHD or HvGD (e.g. after transplantation). The invention comprises also a method for the treatment of a patient suffering from such GvHD and HvGD.

The invention also provides the use of an antibody according to the invention in an effective amount for the manufacture of a pharmaceutical agent, preferably together with a pharmaceutically acceptable carrier, for the treatment of a patient suffering from inflammatory mediator release mediated by CCR5.

The term “graft rejection” as used within this application denotes the response of the human immune system to transplanted tissue. If tissue is transplanted from a donor to a host the human leukocyte antigen genes of the donor's tissue are likely to be different from those of the host's tissue. Thus, the host's immune system recognized the transplanted tissue as foreign and effects an immune response called graft rejection. This graft rejection reaction is called “graft versus host disease” (GvHD).

The term “the sugar chains show characteristics of N-linked glycans attached to Asn297 of an antibody binding to CCR5 recombinantly expressed in a CHO cell” denotes that the sugar chain at Asn297 of the antibody according to the invention has the same structure and sugar residue sequence except for the fucose residue as those of an anti-CCR5 antibody expressed in unmodified CHO cells, e.g. as those anti-CCR5 antibodies according to WO 2006/103100.

The term “NGNA” as used within this application denotes the sugar residue N-glycolylneuraminic acid.

Thus, the current invention provides a method of treating or preventing acute and chronic organ transplant rejection in a mammal, including a human, characterized in administering to said mammal an antibody according to the invention. Also is provided an antibody according to the invention for the treatment or prevention of acute and chronic organ transplant rejection in a mammal, including a human.

Antibody Deposition

| Cell line | Deposition No. | Date of Deposit |
|---|---|---|
| m<CCR5>Pz03.1C5 | DSM ACC 2683 | 18 Aug. 2004 |

The following examples, figures and sequence listing are provided to aid the understanding of the present invention, the true scope of which is set forth in the appended claims. It is understood that modifications can be made in the procedures set forth without departing from the spirit of the invention.

### FIGURES

FIG. 1: Fc receptor binding on CHO cells, afucosylated MAb (□), wt MAb (▪).

FIG. 2: ADCC, afucosylated MAb (▪), wt MAb () and LALA mutant antibody (▴).

FIG. 3: In vivo depletion of CCR5+ cells.

FIG. 4: Monitoring of CCR5 cell expression across CD8+ cells, CD4+ cells and monocytes. Grey: control, unspecific monoclonal antibody; black: treated Cynomolgus

FIG. 5: Afucosylated antibody serum levels (μg/ml) in monotherapy group. Two animals were treated with afucosylated antibody monotherapy at 10 mg/kg by daily intravenous injections on days −1, 5, 8, 14, 21 and 28 or until graft explant.

FIG. 6: Afucosylated antibody serum levels (μg/ml) in combination therapy group. Three additional animals (A: MB021; B: MB106; C: MB116) received afucosylated antibody at 20 mg/kg on days −1, 5, 8, 14, and weekly thereafter until 90 days, with additional Cyclosporine A.

### EXAMPLES

### Antibodies

As antibodies, IgG1 antibody against CCR5 described in WO 2006/103100 and PCT/EP2007/008312 were used (variable regions see SEQ ID NO: 06 to SEQ ID NO: 13, CDR sequences see SEQ ID NO: 04, 05, and SEQ ID NO: 21 to 35). The antibodies used were a wildtype antibody (WT Ab, 8% afucosylated), afucosylated antibody (afucosylated Ab) and LALA mutant antibody (see PCT/EP2007/008312) (LALA mutant Ab). The term “LALA” denotes the amino acid exchange from leucine to alanine at positions 234 and 235 in the constant region of said antibody (L234A, L235A).

### Plasmids

The expression system comprises the CMV promoter system (EP 0 323 997) and is described in tables 1 and 2.

TABLE 1
pETR 3928 (Antibody expression vector)

| Element | Length | Description |
|---|---|---|
| CCR5 HC | 1404 | encoding heavy chain of |
| CCR5 LC | 714 | encoding light chain of |
| GS | 1122 | encoding glutamine-synthetase |
| SV40E | 343 | Promoter |
| hCMV promoter | 1142 | Promoter |
| Intron | 947 | Intron |

TABLE 2
pETR 2896 (Glycosylation vector)

| Element | Length | Description |
|---|---|---|
| chimeric MPSV promoter | 875 | promoter |
| (contains enhancer of | | |
| hCMV promoter) | | |
| synthetic intron | 1324 | intron |
| GnTIII | 1644 | encoding N-acetylglucosaminyl- |
| | | transferase III |
| ManII | 3435 | Encoding mannosidase II |
| pac | 600 | encoding puromycin |
| | | acetyltransferase |
| | | from Streptomyces alboniger |
| polyA | 49 | polyadenylation signal |

### Human CCR5 Cell Line

The L1.2 cell line stably expressing human CCR5 receptor (L1.2hCCR5 cells) are used for the human CCR5 chemotaxis assay. L1.2hCCR5 cells are cultured in RPMI 1640 containing 10% FBS, 10 units/ml Penicillin, 10 μg/ml Streptomycin, 0.1 mM Glutamine, 1 mM Sodium Pyruvate, 55 □M 2-Mercaptoethanol, 250 μg/ml Geneticin (all from Invitrogen). Just prior to the set up of the chemotaxis assay, the cells are spun down and resuspended in Chemotaxis Buffer (Hank's Balanced Salt Solution HBSS (Invitrogen) containing 0.1% BSA (Sigma) and 10 mM HEPES buffer (Invitrogen Corp., USA)). The cells are used in the chemotaxis assay at a final concentration of 5×10⁶ cells/ml.

### Cynomologus CCR5 Cell Line

The L1.2 cell line stably expressing cynomologus CCR5 receptor (L1.2cynoCCR5 cells) are used for the cynomologus CCR5 chemotaxis assay. L1.2cynoCCR5 cells are cultured in the same growth media as the L1.2hCCR5 cells. Cells are seeded at a density of 8×10⁵ cells/ml in growth media containing 5 mM Sodium Butyrate (Sigma) on the day prior to the assay. Just prior to the set up of the chemotaxis assay, the cells are spun down and resuspended in Chemotaxis Buffer. The cells are used in the chemotaxis assay at a final concentration of 5×10⁶ cells/ml.

### Preparation of Ligands

CCR5 ligands human MIP1α, MIP1β or RANTES (R&D Systems) are diluted in Chemotaxis Buffer and are used at a final concentration of 10 nM. LALA mutant Ab and Isotype control mouse IgG2_{A} (BD Biosciences) are diluted in Chemotaxis Buffer.

### Manufacture of Anti-CCR5 Antibodies

Plasmid pETR 3928 was transfected in a glutamine prototroph CHO or HEK293 host cell (EP 0 256 055) which was previously transfected with plasmid pETR 2896. The cell line was cultivated as fed batch cultivation for up to 14 days in serum free medium to generate antibody batches with different amounts of fucosylation (samples 1-4, CHO). The antibody was isolated from the supernatant and purified by chromatographic methods.

WT antibody (fucosylation of 92%) or LALA mutant Ab is recombinantly produced in a HEK 293 or Chinese hamster ovarian (CHO) cell line, CHO-DG44 (Flintoff, W. F., et al., Somat. Cell Genet. 2 (1976) 245-261; Flintoff, W. L., et al., Mol. Cell. Biol. 2 (1982) 275-285; Urlaub, G., et al., Cell 33 (1983) 405-412; Urlaub, G., et al., Somat. Cell Mol. Genet. 12 (1986) 555-566). CHO-DG44 cells were grown in MEM alpha Minus Medium (Gibco No. 22561), 10% dialyzed FCS (Gibco No. 26400-044) and 2 mmol/L L-Glutamine, 100 μM Hypoxanthine, 16 μM Thymidine (HT supplement).

### Example 1

### Binding of Anti-CCR5 Antibodies to CCR5

The binding capability of afucosylated antibody (Ab) and WT anti-CCR5 antibody are compared. The murine L1.2 hCCR5 cell line was used as target cell line. As secondary antibody: FITC-conjugated AffiniPure F(ab)2 Fragment goat anti-human IgG Fcγ specific (Jackson ImmunoResearch Lab # 109-096-098) was used. Anti-human CCR5-FITC (Becton-Dickinson, BD 555992) and mouse IgG2a-FITC were used as control antibodies. RPMI 1640 medium+10% FCS+1% Glutamine+1% Sodium Pyruvate+0.05 mM β-Mercaptoethanol+0.8 mg/ml G418 was the cell culture medium. To induce hCCR5 expression on the cell surface, 0.2 Mio-0.5 Mio cells/ml were incubated in medium containing 1 mM sodium butyrate (Sigma B5887). Cells incubated without sodium butyrate served as negative controls.

### Method:

0.2 Mio cells/180 μl/well diluted in PBS/0.1% BSA were plated in a 96-round bottom plate and 20 μl of diluted antibody was added. After 30 min of incubation at 4° C., cells were washed with PBS/0.1% BSA and 15 μl/well diluted secondary antibody or controls were added. The cells were incubated for another 30 min at 4° C. followed by two washing steps. Before measuring the cells in the FACSCanto, propidium iodide was added.

### Results:

WT Ab and afucosylated Ab showed similar binding on the target cells which was dependent of the antibody concentrations. The EC₅₀ values were calculated for both antibodies using GraphPad Prism 4. The mean values for 100 μg/ml antibody were excluded. The mean values for 100 μg/ml antibody were excluded. EC₅₀ afucosylated Ab: 0.1376; EC₅₀ WT Ab: 0.09407

### Example 2

### Cellular Fc Binding

Fc-binding of afucosylated Ab versus WT Ab was investigated.

CHO cell line expressing ≧10⁴ CCR5 molecules/cell served as a target cell line. As secondary ab:antibody FITC-conjugated AffiniPure F(ab)₂ Fragment goat anti-human IgG F(ab)2 Fragment-specific (Jackson ImmunoResearch Lab # 109-096-097) was used. Anti-human CD16-FITC (Beckman Coulter PN IM0814); mouse IgG1 isotype: mouse IgG1-FITC was used as control. The cell culture medium was IMDM+GlutaMax+25 mM HEPES (Gibco 31980)+10% FCS+HT supplements+6 μM Puromycin. Chinese hamster ovary cells (CHO cells) were cultured in T150 flasks and used for the assay when a density of 13×10⁶ cells/flask was reached. Cells were harvested with Trypsin/EDTA.

### Cell Culture:

medium: IMDM+GlutaMax+25 mM HEPES (Gibco 31980)+10% FCS+HT supplements+6 μM Puromycin Chinese hamster ovary cells (CHO cells) were cultured in T150 flasks and used for the assay when a density of 13 Mio cells/flask was reached. Cells were harvested with Trypsin/EDTA.

### Method:

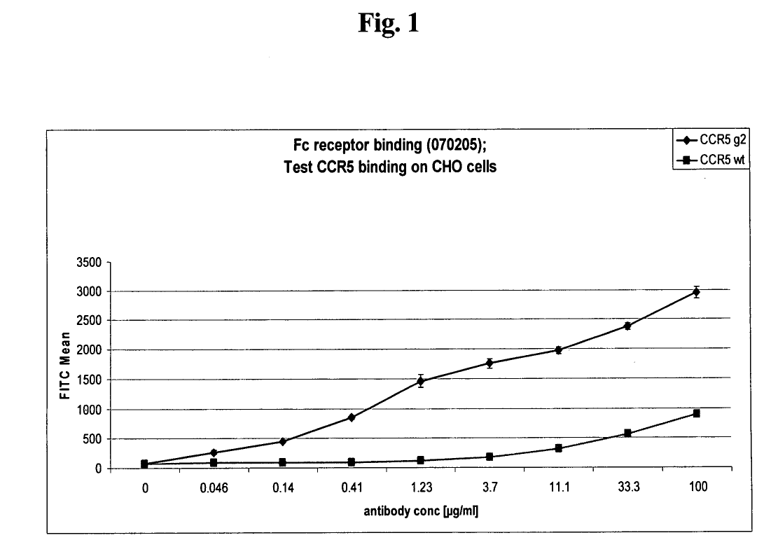

0.2 Mio cells/180 μl/well diluted in PBS/0.1% BSA were plated in a 96-round bottom plate and 20 μl of diluted antibody was added. After 30 min of incubation at 4° C., cells were washed with PBS/0.1% BSA and 12 μl/well diluted secondary antibody or controls were added. The cells were incubated for another 30 min at 4° C. followed by two washing steps. Cells were fixed with 2% PFA for 20 min at 4° C. followed by a washing step before measuring them in the FACSCanto (FIG. 1).

### Example 3

### Determination of the Affinity of Anti-CCR5 Antibodies to FcγIII (CD16a)

His-CD16a was amine coupled to the surface of a CM5-chip. The measurement was performed on a Biacore3000 instrument. The running and dilution buffer was HBS-P. The chip surface was saturated with His-CD16a. Amine coupling groups were saturated. The analyte was added to the buffer flow at a constant concentration of 10 nM, whereas the inhibitor, soluble CD16a was added to the buffer flow at increasing concentrations (0-1000 nM). RU values reflect the affinity between antibody and CD16a.

TABLE 3

| | RU max | IC50 [nM] |
|---|---|---|
| WT Ab | 48 ± 1 | 14 ± 1 |
| LALA mutant Ab | no binding | no binding |
| Ab according to | 102 ± 8 | 9 ± 1 |
| the invention | | |
| Positive Control | 32 ± 1 | 10 ± 0 |
| Negative Control | no binding | no binding |

### Example 4

Potential of anti-CCR5 monoclonal antibodies to bind to FcγRIIIa on NK cells

To determine the ability of the antibodies of the invention to bind to FcγRIIIa (CD16) on Natural Killer (NK) cells, Peripheral Blood Mononuclear Cells (PBMCs) are isolated and incubated with 20 μg/ml of antibody and control antibodies in the presence or absence of 20 μg/ml of a blocking mouse antibody to FcγRIIIa (anti-CD16, clone 3G8, RDI, Flanders, N.J.), to verify binding via FcγRIIIa. As negative controls, human IgG2 and IgG4 (The Binding Site), that do not bind FcγRIIIa, are used. Human IgG1 and IgG3 (The Binding Site) are included as positive controls for FcγRIIIa binding. Bound antibodies on NK cells are detected by FACS analysis using a PE-labeled mouse anti-human CD56 (NK-cell surface marker) antibody (BD Biosciences Pharmingen, San Diego, Calif.) in combination with a FITC-labeled goat F(ab)₂ anti-human IgG (Fc) antibody (Protos Immunoresearch, Burlingame, Calif.). Maximum binding (Bₘₐₓ) is determined at an antibody concentration of 20 μg/ml. Control antibody (human IgG4) shows up to 30% Bₘₐₓ compared to 100% Bₘₐₓ for human IgG1. Therefore “no FcγRIIIa binding or no ADCC” means at an antibody concentration of 20 μg/ml a Bₘₐₓ value of up to 30% compared to human IgG1.

### Measurement of ADCC and CDC by Chromium⁵¹-Release Assay

### Materials:

Chromium from Amersham, Cat # CJS11; Round bottom polypropylene plates costar, Cat # 3790; Lumaplate-96 (Solid scintillator coated polystyrene plates): From Perkin-Elmer, Part #-6006633.

Target Cells: CCR5-expressing cells (L1.2 cell line, see example 2).

Effector cells or Complement serum (human PBMCs, isolated NK cells)

### Method:

1×10⁶ target cells are labeled with 100 μCi of Chromium⁵¹ for 1 hour at 37° C. Labeled cells are washed four times with the medium and resuspended in 5 ml (concentration 200,000 cells/ml). 50 μl of target cells (at a concentration of 200,000 cells/ml) are plated per well. 50μl of test antibody is added at different concentration and incubated at 4° C. for 1 hour. Effector cells (at desired ratio) or Complement serum at the desired E:T ratio was added and cells are incubated at 37° C. for 4 hours.

### Controls:

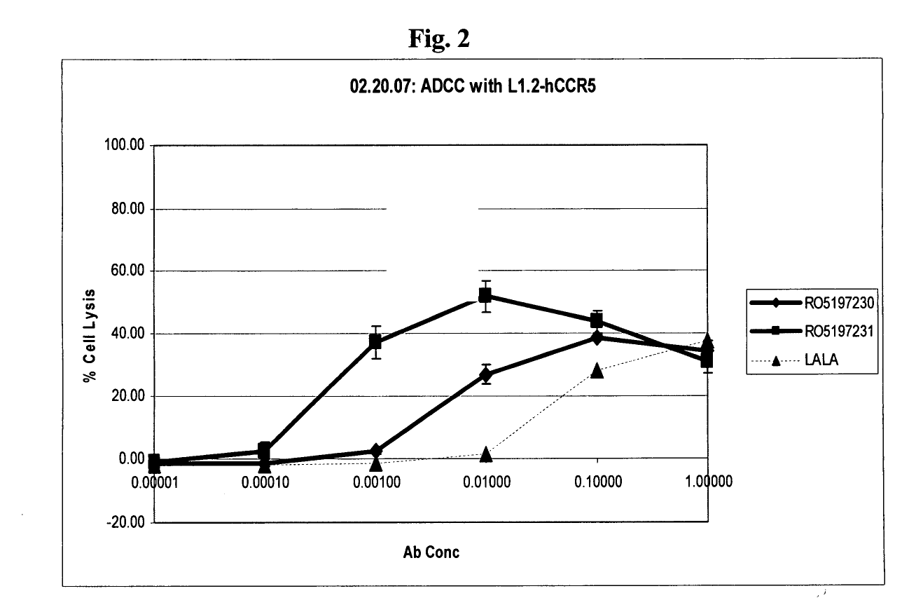

For Spontaneous lysis control, 50 μl target cells and 100 μl culture media was mixed and measured. Maximum lysis was measured using 50 μl labeled target cells+50 μl Triton-X-100 (1% in PBS)+50 μl media At the end of incubation 50 μl of culture supernatant was added on the Luma plate. Results were readed in a top count. Cytotoxicity was calculated using percent cytotoxicity equals sample CPM—spontaneous lysis CPM divided by maximal lysis CPM—spontaneous lysis CPM multiplied by 100. Results are shown in FIG. 2.

### Example 5

### CCR5 Chemotaxis Assay

L1.2hCCR5 cells harboring the human or L1.2mCCR5 harboring the cynomologous CCR5 are cultured in RPMI 1640 containing 10% Fetal bovine serum, 1×Penicillin/Streptomycin, 1×Glutamine, 1× Sodium Pyruvate, 1×β-Mercaptoethanol, and 250 μg/ml G418 (all from Invitrogen). Just prior to the set up of the chemotaxis assay, the cells are spun down and resuspended in Chemotaxis Buffer (Hank's Balanced Salt Solution HBSS (Invitrogen) containing 0.1% BSA and 10 mM HEPES). The cells are used in the chemotaxis assay at a final concentration of 5×10⁶ cells/ml. CCR5 ligands hMIP 1α, hMIP 1β or hRANTES (R&D Systems) are diluted in Chemotaxis Buffer and are used at a final concentration of 20 nM. Test antibodies or the appropriate isotype control antibodies are diluted in HBSS. Chemotaxis is set up in the 0.5 μm pore 96-well ChemoTx^{R} system (Neuroprobe). Each antibody is mixed with one of the CCR5 ligands and 30 μl of this mixture is placed in the bottom well of the ChemoTx^{R} system. The filter screen in placed on top of the bottom wells. Each antibody is mixed with the L1.2hCCR5 or L1.2mCCR5 cells and 20 μl of this mixture is placed on the filter. The plates are then placed in a humidified chamber and incubated at 37° C. and 5% CO₂ for 3 hours. After incubation, the cells are scraped off the filter and the plates are spun in a table top centrifuge at 2,000 rpm for 10 min. The filter is then removed and the density of the cells that have migrated to the bottom wells is detected using CyQUANT® Cell proliferation assay kit (Invitrogen) and the Spectra MAX GeminiXS plate reader (Molecular Devices) according to the manufacturers' instructions. IC₅₀ is calculated using Prism 4 (GraphPad).

L1.2CCR5 cells were seeded at 8×10⁵ cells/ml with 2 mM Sodium Butyrate 24 h before assay. Antibodies were diluted in CTX buffer (HBSS, 0.1% BSA, 10 mM HEPES). Ligands were diluted (MIP1a MIP1b RANTES, 20 nM stock (2×) in CTX buffer). Cells were washed and resuspended in CTX buffer (HBSS, 10 mM HEPES, 0.1% BSA) at 1×10⁷ (2×). In deep well blocks were prepared: antibody+ligand and top suspension antibody+cells. The assay was set up into chemotaxis onto 101-5 ChemoTxR (Neuro Probe) plates, incubated 3 hrs at 37° C. in a humidified chamber. Wash-Scrape cells off top of filter and spin plates 2,000 rpm for 10 min. Filter was removed and 10 ml supernatant from each bottom well was taken. After freezing/thawing 10 ml 2×CyQUANT (Invitrogen) was added to each well and results were read on a fluorescent plate reader.

### Results:

Migration of human CCR5 cells was effectively blocked by WT Ab and afucosylated Ab (table 4).

TABLE 4

| | Chemotaxis IC₅₀ (Midpoint, [nM]) | | |
|---|---|---|---|
| Antibody | MIP1a | MIP1b | RANTES |
| LALA mutant Ab | 1.38 ± 0.2 | 1.50 ± 0.6 | 5.04 ± 2.9 |
| WT Ab | 0.66 ± 0.3 | 0.44 ± 0.0 | 1.48 ± 0.2 |
| Afucosylated Ab | 0.67 ± 0.1 | 2.36 ± 1.4 | 0.55 ± 0.01 |

### Example 6

### In Vivo Depletion of CCR5⁺ Cells

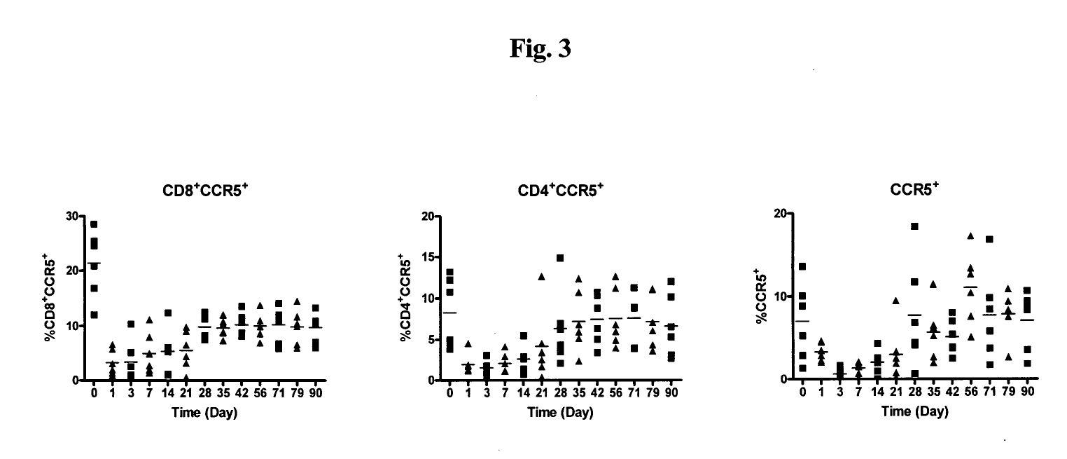

Cynomolgus monkeys received 1 single i.v. infusion of 1 mg/kg or 10 mg/kg afucosylated Ab. Data were shown as % of CCR5+ cells in the indicated subsets in blood (CD8+ and CD4+ T-cells as well as monocytes) (FIG. 3).

### Example 7

### In Vivo Depletion Toxicity Study

Purpose: To monitor CCR5 cell expression across CD8+ cells, CD4+ cells and monocytes. Determine if CD8+ cells are depleted in animals treated with afucosylated Ab to determine effect of treatment on CCR5 expression in the tissues.

12 Animals: 4 control, 8 with 21 mg/kg/day

Dose on Day 1 and Day 15

Whole blood for staining:

Pre-dose Day-6, Pre-dose Day 1, 2, 4, 8, 15, 16, 18, 22, 29, 36, 43, 50, 57, 64, 71.

Stain for CCR5+ in CD8+, CD4+, CD8+CD4+, Monocytes (by gate)

Stain for CXCR3+ in CD8+ and CD4+

Depletion confirmation by counting beads

Spleen, lymph node and bone marrow for staining CCR5 on Day 8, 15, 18, 71.

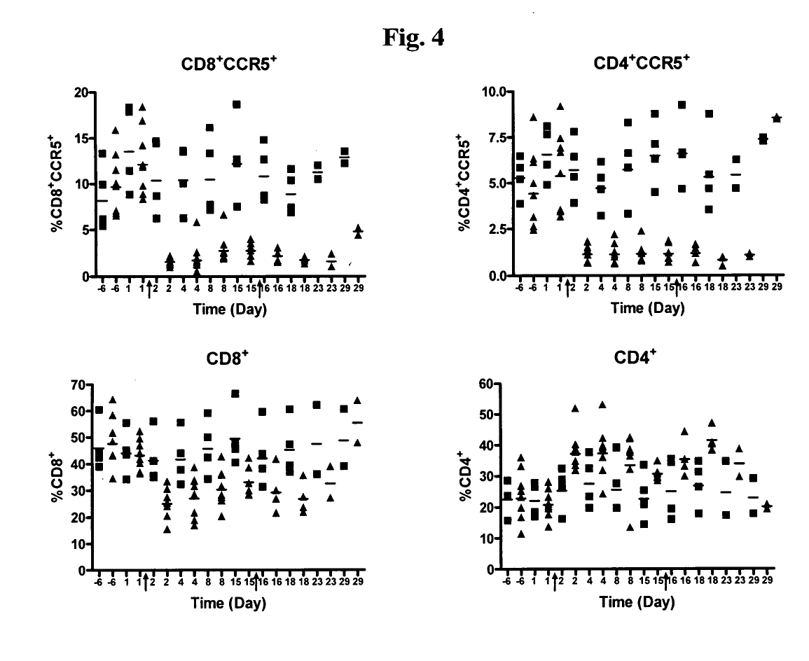

The results are shown in FIG. 4.

### Example 8

### Analysis of Glycostructure of Antibody

For determination of the relative ratios of fucose- and non-fucose (afucose) containing oligosaccharide structures, released glycans of purified antibody material were analyzed by MALDI-Tof-mass spectrometry. For this, the antibody sample (about 50 μg) was incubated over night at 37° C. with 5 mU N-Glycosidase F (Prozyme# GKE-5010B) in 0.1 M sodium phosphate buffer, pH 6.0, in order to release the oligosaccharide from the protein backbone. Subsequently, the glycan structures released were isolated and desalted using NuTip-Carbon pipette tips (obtained from Glygen: NuTip1-10 μl, Cat.Nr#NT1CAR). As a first step, the NuTip-Carbon pipette tips were prepared for binding of the oligosaccharides by washing them with 3 μL 1 M NaOH followed by 20 μL pure water (e.g. HPLC-gradient grade from Baker, #4218), 3 μL 30% (v/v) acetic acid and again 20 μl pure water. For this, the respective solutions were loaded onto the top of the chromatography material in the NuTip-Carbon pipette tip and pressed through it. Afterwards, the glycan structures corresponding to 10 μg antibody were bound to the material in the NuTip-Carbon pipette tips by pulling up and down the N-Glycosidase F digest described above four to five times. The glycans bound to the material in the NuTip-Carbon pipette tip were washed with 20 μL pure water in the way as described above and were eluted stepwise with 0.5 μL 10% and 2.0 μL 20% acetonitrile, respectively. For this step, the elution solutions were filled in a 0.5 mL reaction vials and were pulled up and down four to five times each. For the analysis by MALDI-Tof mass spectrometry, both eluates were combined. For this measurement, 0.4 μL of the combined eluates were mixed on the MALDI target with 1.6 μL SDHB matrix solution (2.5-dihydroxy-benzoic acid/2-hydroxy-5-methoxybenzoic acid [Bruker Daltonics #209813] dissolved in 20% ethanol/5 mM NaCl at 5 mg/ml) and analyzed with a suitably tuned Bruker Ultraflex TOF/TOF instrument. Routinely, 50-300 shots were recorded and summed up to a single experiment. The spectra obtained were evaluated by the flex analysis software (Bruker Daltonics) and masses were determined for the each of the peaks detected. Subsequently, the peaks were assigned to fucose or afucose (non-fucose) containing glycostructures by comparing the masses calculated and the masses theoretically expected for the respective structures (e.g. complex, hybride and oligo- or high-mannose, respectively, with and without fucose).

For determination of the ratio of hybride structures, the antibody sample was digested with N-Glycosidase F and Endo-Glycosidase H concomitantly. N-glycosidase F releases all N-linked glycan structures (complex, hybride and oligo- and high mannose structures) from the protein backbone and the Endo-Glycosidase H cleaves all the hybride type glycans additionally between the two GlcNAc-residues at the reducing end of the glycan. This digest was subsequently treated and analyzed by MALDI-Tof mass spectrometry in the same way as described above for the N-Glycosidase F digested sample. By comparing the pattern from the N-Glycosidase F digest and the combined N-glycosidase F/Endo H digest, the degree of reduction of the signals of a specific glycostructure is used to estimate the relative content of hybride structures.

The relative amount of each glycostructure was calculated from the ratio of the peak height of an individual glycol structure and the sum of the peak heights of all glycostructures detected. The relative amount of afucose is the percentage of fucose-lacking structures related to all glycostructures identified in the N-Glycosidase F treated sample (e.g. complex, hybrid and oligo- and high-mannose structures, resp.), see table 5.

TABLE 5
Afucosylation of MAbs

| Sample | Afucosylation (in %) |
|---|---|
| No. 1 | 70.0 |
| No. 2 | 72.8 |
| No. 3 | 72.5 |
| No. 4 (HEK293) | 57 |

### Example 9

### Anti-CCR5 Antibody-Based Immunosuppressive Regimen in a Model of Heart Transplantation in Non-Human Primates (Cynomolgus Monkeys).

The efficacy of an afucosylated antibody of the invention was demonstrated in depleting CCR5+ cells in Cynomolgus monkeys receiving a heart transplant from a mismatched donor animal. The number of CCR5+ cells is measured in the graft 4-5 days after transplant. In addition the immunosuppressive effect of the afucosylated antibody was evaluated as monotherapy, as measured by the duration of allograft survival.

Animals: Cynomolgus monkeys (Macaca fascicularis) (3 to 7 Kg) were paired with blood type compatible, mixed lymphocyte reaction (MLR)-mismatched animals (actual SI range: 6-8). Animals were housed under conventional conditions and used according to the guidelines of the Institutional Animal Care and Use Committee (IACUC) of the University of Maryland Medical School. Protocols approved by the IACUC were carried out in compliance with the Guide for the Care and Use of Laboratory Animals (HHS, NIH Publication 86-23, 1985).

Cardiac Allograft: All recipient animals underwent heterotopic intraabdominal cardiac allograft transplantation, as described previously (C. Schroeder et al., J. Immunol. (2007) 179:2289-99). Two animals were treated with afucosylated antibody monotherapy at 10 mg/kg by daily intravenous injections on days −1, 5, 8, 14, 21 and 28. Three additional animals received afucosylated antibody at 20 mg/kg on days −1, 5, 8, 14, and weekly thereafter until day 90, and with additional Cyclosporine A (CsA, generic formulation from Bedford Laboratories, Bedford, Ohio). CsA was given once daily (IM at 15±10 mg/kg) from the day of surgery until day 90 to achieve target trough levels (>400 ng/ml). Animals experiencing acute rejection episodes received a 3-day course of steroids (10 mg/kg boluses, Solu-Medrol®, Pharmacia, Kalamozoo, Mich.) to attain graft survival of 90 days. Open cardiac biopsies were performed 30 minutes after graft revascularization and on postoperative days 5 (monotherapy only), 14, 28 and 56. Graft function was monitored at least daily by implanted telemetry (Data Sciences International, St. Paul, Minn.). Clinical acute graft rejection was suspected based on two of three cardinal signs: consistent high body temperature (>38.5° C.); a decrease in graft heart rate (to <120 beats per min (bpm), or a sustained drop of >40 bpm (˜20%) from a stable baseline); or a decrease in graft pulse pressure (systolic minus diastolic) of >20 mmHg not attributable to technical measurement issues. Graft failure was defined as loss of contraction by telemetry and confirmed by visualization at explant, and was always preceded by signs of acute rejection. Body temperature was measured using the DSI telemetry system daily and recorded as a single morning measurement. Reference animals include historical animals receiving either no treatment (n=5) or CsA dosed to achieve target trough levels >400 ng/ml (“therapeutic” CsA treatment, n=8).

CBC and FACS analyses: Complete blood cell (CBC) assays were performed on freshly collected EDTA-blood using an automated cell counter (Hemavet) using monkey settings. Whole blood collected in EDTA (100 μl), and cells isolated from lymph node (LN) (1×10⁶ cells) were analyzed for expression of CCR5 at regular intervals. Briefly, cells were stained for 20 min at 4° C. with PerCP-Cy5.5-conjugated anti-human CD4 mAb (L200, BD Pharmingen, San Diego, Calif.), APC-conjugated anti-human CD8α (3B5, Caltag, InVitrogen, Carlsbad, Calif.), AlexaFluor488-conjugated anti-human CD14 (M5E2, BD Pharmingen) and PE-conjugated anti-human CD195 (CCR5) (3A9, BD Pharmingen) in FACs wash buffer (PBS supplemented with 10% FCS and 0.2% sodium azide). Red blood cells were lysed with BD FACSLyse. In some experiments, cells were also stained for CXCR3 with AlexaFluor488-conjugated anti-human CD183 (CXCR3) (1C6, BD Pharmingen) and in two instances graft infiltrating cells (GILs) were isolated by collagenase digestion and Ficoll gradient separation and stained as above. Lymphocyte populations were gated by forward/side scatter analysis to exclude debris. Data analysis and graphic display were conducted using CellQuest or Winlist software. The proportion of CCR5-positive cells among CD4+ or CD8+ lymphocytes in the blood was multiplied by absolute counts of CD4 and CD8 calculated from lymphocytes counts from the differential analysis to obtain absolute counts of CCR5+CD4+ and CCR5+CD8+ cells per μl of blood.

Detection of anti-donor alloantibody: Alloantibodies were measured retrospectively by flow cytometry as described previously (Schroeder et al., supra). Briefly, archived frozen donor splenocytes (0.5×10⁶ cells) were incubated with heat-inactivated recipient serum (50 μl) for 30 min at 4° C. After washing, antibody binding was revealed using PE-labeled goat anti-human IgM (Fcγ specific) antibodies (Biosource, Invitrogen, Carlsbad, Calif.), or biotin-labeled goat anti-monkey IgG (Fcγ specific) antibodies (Nordic, Tilburg, The Netherlands) followed by PE-labeled streptavidin (BD Pharmingen, San Diego, Calif.). FITC-labeled anti-human CD3 (BD Pharmingen) was added to gate T cells. Data were expressed as the calculated percentage of T cells positive post-transplant after subtraction of pre-transplant levels. Reactivity was defined as an increase of more than 10%. Two animals in CsA group were not evaluated due to absent viable donor cells.

Histology: Tissue was fixed with 10% formalin and processed routinely for paraffin embedding. Sections of paraffin-embedded tissue were stained with hematoxylin and eosin. Cellular infiltrates were graded for acute rejection by ISHLT criteria. CAV incidence in beating hearts explanted after day 70 was recorded as percent of arteries and arteriolar vessels involved (CAV score ≧1) at each time point. CAV severity was scored in these explanted hearts as follows: Grade 0, normal arterial morphology; Grade 1, activated endothelial cells with enlarged nuclei and/or adherent leukocytes, without luminal narrowing (<10%); Grade 2, distinct neointimal thickening, luminal narrowing <50%; Grade 3, extensive neointimal proliferation with greater than 50% luminal occlusion. Scoring was independently performed for each explanted heart by three evaluators (TS, RNP, TZ) blinded with respect to treatment group. The mean CAV score for each biopsy or explant was calculated using the equation: (#grade O−vessels×0)+(#grade 1−vessels×1)+(#grade 2−vessels×2)+(#grade 3−vessels×3)/total number of arterial vessels scored. Individual graft mean CAV scores were averaged to calculate the group mean (±SD) for each treatment group.

Immunohistochemistry: Immunohistochemical stains were performed using an automated method. Formalin fixed paraffin embedded (FFPE) tissue sections were deparaffinized and stained on a Ventana ES automated stainer using the ABC method (Ventana Medical Systems, Inc., Tucson, Ariz.). All reagents placed on the Ventana instrument were purchased from Ventana. Settings were adjusted on mild CC1, conditioner 1 and standard 1. The following primary antibodies were used: CD3 (2GV6, Ventana), CD68 (KP1, DAKO) and CD20 (L26, DAKO, Copenhagen, Denmark). For animals treated with afucosylated antibody monotherapy and untreated controls, the number of cells was calculated as follows: the area of tissue with low, moderate or strong cellular infiltration was estimated, then 10 pictures corresponding to representative fields were taken. The number of cells per field was then counted, and the average of cells per field was calculated. If a tissue sample was divided in different blocks, all blocks were processed separately, and the number of cells/field for all blocks was averaged to obtain cell counts for that tissue sample. For animals treated with afucosylated antibody +CsA and CsA controls, cellular infiltration was scored using the following scale: 0, absence of cells; 1, focal staining or weak diffuse; 2, 1-3 nodules or mild diffuse interstitial infiltration; 3, 3-10 nodules or moderate infiltration; 4, >10 nodules or strong infiltration; 5, massive infiltration. FFPE and frozen tissue section blocks were evaluated for CCR5+ cells.

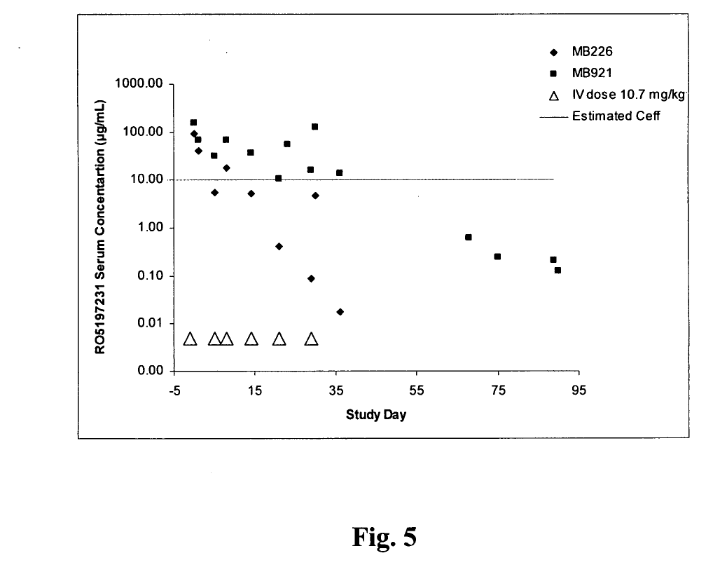

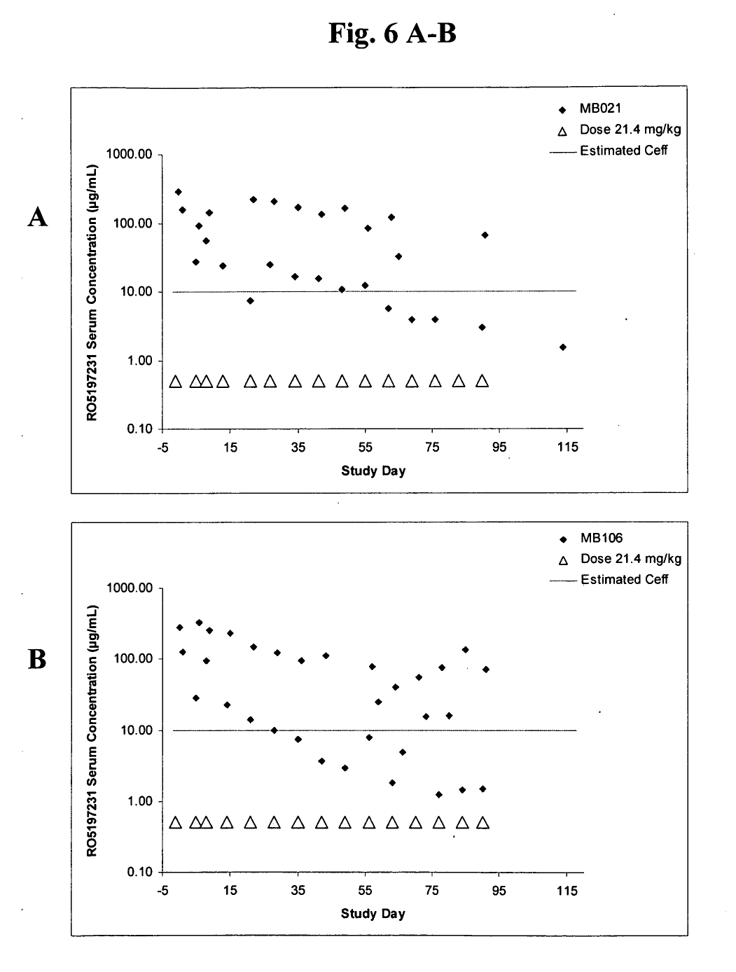

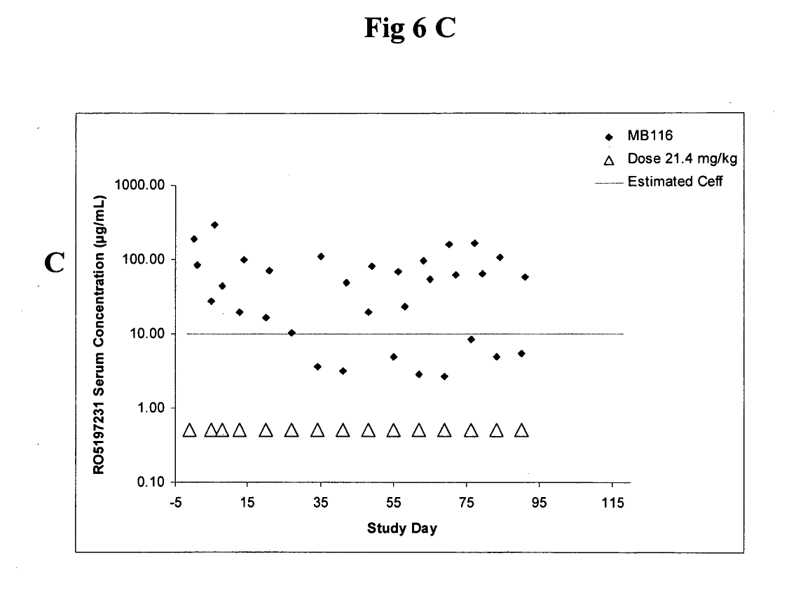

Drug level analysis: Serum samples were collected at respective study time points and shipped to Roche for measurement of afucosylated antibody concentrations. CsA plasma levels were measured by HPLC method. The results are shown in FIG. 5 and FIG. 6.

Statistical analysis: Graft survival time was expressed as median survival time (MST) and graphed with use of the Kaplan-Meier method. The log-rank test was used to compare survival time between different groups. Continuous variables were expressed as the mean plus standard deviation unless otherwise indicated and were compared using the Mann-Whitney non parametric test. Nominal variables (i.e., incidence of early rejection) were measured using a contingency table and the Fischer exact test. P-values less than 0.05 were considered statistically significant. All statistical analyses were performed on a personal computer with the statistical package SPSS for Windows XP (Version 11.0, SPSS, Chicago, Ill., USA) or GraphPad InStat (version 5.1, GraphPad Software, San Diego, Calif., USA).

### Results:

Afucosylated antibody depleted CCR5+ T-cells in blood and lymph nodes: One day following administration of afucosylated antibody, CCR5 expression dropped in both PBL and LN CD4+ and CD8+ cells in all animals, as shown in Table 6. In the lymph nodes of animals treated with afucosylated antibody monotherapy, CCR5 expression remained low on CD8+ cells, whereas it rebound at day 7 on CD4+ cells. In the lymph nodes of animals treated with afucosylated antibody +CsA bitherapy, efficient depletion of CCR5 cells was achieved for the first two weeks in all three animals, after which CCR5 levels remained low in one animal (MB021) whereas they fluctuated in the other two animals (MB106, MB116). In the blood, a rebound in CCR5+ cells was detected at different intervals post-transplant for all three afucosylated antibody +CsA animals. To begin to investigate the reasons for this unexpected rebound in CCR5+ cells despite ongoing therapy, we measured CCR5+ cells one day after the last dose of antibody, on day 91. CCR5 expression on PBL was decreased in association with “peak” afucosylated antibody levels in MB 106, but not in MB021.

TABLE 6
Analysis of CCR5 expression on blood and lymph nodes CD4+ and CD8+
cells by flow cytometry.

| | Cyno | D−7, −1 | Day 0 | Day | Day | Day | Day | Day 91 |
|---|---|---|---|---|---|---|---|---|
| Regimen¹ | ID | (baseline) | (peak)^{#} | 14* | 28* | 56* | 90* | (peak)^{##} |

Blood CD4 (cells/uL)

| | | | | | | | | |
|---|---|---|---|---|---|---|---|---|
| AA (10) | MB226 | 40 | 108 | 132 | 297 | | [79] | |
| AA (10) | MB921 | 87 | 27 | 239 | 66 | | [174] | |
| CsA + AA (20) | MB021 | 413 | 166 | 42 | 288 | 351 | 357 | 439 |
| CsA + AA (20) | MB106 | 113 | 200 | 18 | 39 | 141 | 113 | 44 |
| CsA + AA (20) | MB116 | 237 | 39 | 36 | 22 | 280 | | 59 |

Blood CD8 (cells/uL)

| | | | | | | | | |
|---|---|---|---|---|---|---|---|---|
| AA (10) | MB226 | 320 | 237 | 170 | 715 | | [342] | |
| AA (10) | MB921 | 541 | 60 | 361 | 15 | | [82] | |
| CsA + AA (20) | MB021 | 310 | 99 | 3 | 28 | 578 | 525 | 367 |
| CsA + AA (20) | MB106 | 104 | 88 | 5 | 34 | 383 | 210 | 24 |
| CsA + AA (20) | MB116 | 154 | 15 | 3 | 9 | 352 | | 109 |

LN CD4 (Percent, Average of pieces A and B)

| | | | | | | | |
|---|---|---|---|---|---|---|---|
| AA (10) | MB226 | (2.3 +/− 1) | 0.44 | 0.41 | | | |
| AA (10) | MB921 | (2.3 +/− 1) | 0.64 | 0.96 | | | |
| CsA + AA (20) | MB021 | (2.3 +/− 1) | 0.99 | 0.43 | 0.79 | 0.49 | 0.51 |
| CsA + AA (20) | MB106 | (2.3 +/− 1) | 0.64 | 0.42 | 0.95 | 1.34 | 0.63 |
| CsA + AA (20) | MB116 | (2.3 +/− 1) | 0.64 | 0.37 | 1.37 | 1.84 | 0 |

LN CD8 (Percent, Average of pieces A and B)

| | | | | | | | |
|---|---|---|---|---|---|---|---|
| AA (10) | MB226 | (8 +/− 0 4) | 0.72 | 0.37 | | | |
| AA (10) | MB921 | (8 +/− 0.4) | 0.71 | 0.41 | | | |
| CsA + AA (20) | MB021 | (8 +/− 0.4) | 1.77 | 0.56 | 0.75 | 0.77 | 0.23 |
| CsA + AA (20) | MB106 | (8 +/− 0.4) | 0.48 | 0.48 | 1.57 | 2.83 | 0.42 |
| CsA + AA (20) | MB116 | (8 +/− 0.4) | 0.82 | 0.85 | 6.18 | 3.65 | 0 |

¹CsA: Cyclosporine A given at 15 ± 10 mg/ml daily under graft explant to achieve trough levels >400 ng/ml; AA: treatment with anti-CCR5 antibody afucosylated antibody given at 10 mg/kg on days −1, 5, 8, 14, 21 and 28 or until graft explant (monotherapy) or 20 mg/kg on days −1, 5, 8, 14, and weekly thereafter until 90 days until 90 days (combined therapy)
( ) Data in parenthesis represent the mean ± SD of five donor lymph nodes stained to provide a normal reference range as context for analysis of CCR5% expression in the lymph nodes
^{#,##}CCR5 expression was measured one day after the first and last dose of antibody respectively.
*CCR5 expression was measured weekly just before the next dose of afucosylated antibody, and therefore represents CCR5 expression at afucosylated antibody “trough”.
[ ] Represents CCR5 expression measured after therapy with afucosylated antibody was discontinued.

Graft survival: In cynomolgus macaques treated with afucosylated antibody monotherapy at 10 mg/kg every 3-5 days for the first week and then weekly, cardiac allografts survived for 14 and 23 days, significantly longer than in untreated monkeys (MST 6.5±0.4 days; n=5, p=0.034) (Table 7). Both grafts failed due to clinical acute rejection (decreased contractility, edema, graft enlargement) despite ongoing afucosylated antibody monotherapy. Interestingly, acute rejection occurred sooner in animal MB226 which showed reduction in afucosylated antibody trough levels following repeated dosing (FIG. 5). Acute cellular rejection was confirmed histologically. When CsA was combined with afucosylated antibody therapy dosed as above, none of 3 animals treated with bitherapy developed symptomatic rejection through the end of observation at day 85-90. One animal (MB116) developed hypothermia at day 90. Infection was suspected, and gram negative septicemia was subsequently confirmed by blood culture. The animal was electively sacrificed at the time of explant due to suspected sepsis. No clinical evidence for infectious source (no abscess, cellulitis, pneumonia, or wound infection) was evident at necropsy. Central line infection is therefore presumed to be the source of gram negative septicemia. The two other normally beating, healthy-appearing grafts were also without clinical evidence of rejection and were electively explanted at 90 days. In conclusion, there was no episode of acute graft rejection detected during the treatment with CsA+ afucosylated antibody, and all three grafts were electively explanted with a normal function at day ˜90. In contrast, in four of eight historical CsA-treated animals, a first episode of symptomatic acute allograft rejection (graft bradycardia and/or diminished contractility, recipient fever) was detected before 90 days (at 7, 23, 44 and 71 days respectively). In 3 animals rejection was steroid-responsive (three daily steroid boluses with Solu-Medrol®, 10 mg/kg); one graft rejected on day 7 before treatment could be initiated. One animal (MA029) died at day 26 with a septicemia from a central venous line infection. The remaining 3 grafts survived without acute rejection to elective graft explant at day 85-90. Thus, there is a trend towards prolonged graft survival (median survival time, MST >90d vs 71d with CsA, p=0.13) and diminished incidence of acute rejection (0/3 vs 4/7, p=0.2) with CsA+ afucosylated antibody as compared to CsA alone.

TABLE 7
Graft Outcome and Histology¹

| | | | | Rejection at graft explant | | | |
|---|---|---|---|---|---|---|---|
| | Cyno | 1° | | | CAV | CAY | |
| Regimen | ID | survival | 2° survival | ISHLT | severity | incidence | Notes |

Historical controls

| | | | | | | |
|---|---|---|---|---|---|---|
| Untreated | M360 | 6 | 3A | N/A | N/A | |
| Untreated | M364 | 6 | 3A | N/A | N/A | |
| Untreated | M20 | 6.5 | 3A | N/A | N/A | (2) |
| Untreated | M278 | 6.5 | 3B | N/A | N/A | |
| Untreated | M342 | 7 | 3B | N/A | N/A | |

Current study

| | | | | | | |
|---|---|---|---|---|---|---|
| AA (10) | MB226 | 14 | N/A | 3R | 0.75 | 54% |
| AA (10) | MB921 | 23 | N/A | 3R | 2.33 | 98% |

Historical controls

| | | | | | | | |
|---|---|---|---|---|---|---|---|
| CsA | M162 | 7 | | 4 | N/A | N/A | |
| CsA | MA029 | >26 | N/A | | | | (3) |
| CsA | MA939 | 44 | >47 | | 1.5 | | (4) |
| CsA | M9421 | 23 | 72 | 2 | 2.27 | 97% | |
| CsA | M115 | 71 | >92 | 4 | 2.45 | 100% | |
| CsA | M262 | >85 | N/A | 2/3A | 1.55 | 67% | |
| CsA | MA095 | >89 | N/A | 3A | 1.5 | 85% | |
| CsA | MA049 | >91 | N/A | 2 | 2.1 | 95% | |

| | | |
|---|---|---|
| CAV at 70-90 days | 1.9 ± 0.4 | 89 ± 12 |

Current study

| | | | | | | | |
|---|---|---|---|---|---|---|---|
| CsA + AA (20) | MB021 | >92 | N/A | 1R | 0.26 | 20% | |
| CsA + AA (20) | MB106 | >91 | N/A | 0 | 0.07 | 7% | (5) |
| CsA + AA (20) | MB116 | >91 | N/A | 0 | 0.2 | 10% | (6) |

| | | |
|---|---|---|
| CAV at 90 days | 0.2 ± 0.1 | 12 ± 7 |

¹Secondary survival indicates the time at which the rejected graft was explanted after a first episode of acute rejection was treated; >: indicates a graft explanted while still beating; CAV: cardiac allograft vasculopathy; ISHLT: rejection score according to the International Society of Heart and Lung Transplantation; CsA: Cyclosporine A given at 15 ± 10 mg/ml daily under graft explant to achieve trough levels >400 ng/ml; AA: treatment with anti-CCR5 antibody afucosylated antibody given at 10 mg/kg on days −1, 5, 8, 14, 21 and 28 or until graft explant (monotherapy) or 20 mg/kg on days −1, 5, 8, 14, and weekly thereafter until 90 days until 90 days (combined therapy)
(2) Death due to hemorrhage
(3) Death due to infection
(4) Death due to bowel necrosis
(5) Lymphoma at day 90
(6) Infection at day 90 (aerobic GNR). Cellular infiltration in some tissues.

Histology: Allografts rejecting under monotherapy exhibited typical features of severe acute cellular rejection (Grade 3R, with diffuse inflammatory infiltration with multifocal cardiac myocyte damage and associated edema and hemorrhage) according to the International Society of Heart and Lung Transplantation (ISHLT) criteria. In contrast, rejection scores were consistently lower (Grade 0 or 1) on protocol biopsies and at graft explant in monkeys treated with afucosylated antibody +CsA versus afucosylated antibody alone or no treatment. (Table 7). Remarkably, whereas all grafts treated with CsA monotherapy exhibited moderate to severe cardiac allograft vasculopathy (CAV severity score 1.9±0.4; all scores ≧1.5, N=6) at explant, the CAV score associated with CsA+ afucosylated antibody was significantly reduced relative to therapeutic CsA alone (0.2±0.1, n =3; p=0.024 vs CsA). Cellular infiltrate usually included large numbers of CD3 and CD68. Interestingly, T cell (CD3) infiltration tended to be lower in animals with the lowest CCR5 expression on blood cells at day 91 (Table 6).

Alloantibody: Alloantibody elaboration was consistently detected within two weeks in both animals treated with CCR5 depletion alone. We conclude that afucosylated antibody (10.7 mg/kg) monotherapy does not significantly modulate alloantibody elaboration in response to a cardiac allograft. When a higher dose of afucosylated antibody (21.4 mg/kg) was administered with CsA, two of three animals elaborated only trace/measurable anti-donor IgG antibody with none reaching the positivity threshold of 10%, and only one of these also exhibited transient low-titer IgM at one month (MB106). In contrast, alloantibody production was detected within 90 days in 6/7 (IgM) and 4/7 (IgG) historical control animals treated with CsA alone. We conclude that additional treatment with afucosylated antibody significantly attenuated alloantibody elaboration in response to a cardiac allograft in the context of concomitant calcineurin inhibitor therapy.

Body temperature: The rise in body temperature typically observed in the first 3 days in all untreated control animals was not prevented with afucosylated antibody monotherapy. The later rise in body temperature normally seen in acute rejection was blocked in one animal (MB021). Temperature was similar in CsA-treated animals independent of additional treatment with afucosylated antibody.

Drug trough levels and immunogenicity: CsA was dosed at 15±10 mg/kg IM daily to achieve therapeutic trough levels >400 ng/ml. CsA coverage achieved was similar between groups, with CsA blood levels of 569±310 ng/ml (average of mean ±SD, n=4 evaluated) in historical controls and 547±327 ng/ml (average of mean ±SD, n=3) in animals receiving CsA +afucosylated antibody. The concentrations of afucosylated antibody reached >100 μg/ml (Cmax) 24 hrs after the first intravenous dose of 10.7 mg/kg. In animal MB226, afucosylated antibody trough levels decreased with repeated dosing and this recipient rejected its cardiac allograft by day 15. Production of anti-drug antibodies was suspected to be the cause but this was not verified.

Complications and clinical assessment: afucosylated antibody infusion was well-tolerated, without evidence for clinical illness during acute infusion or associated with ongoing treatment, alone or in combination with CsA. Assessment of drug toxicity or PK was not an aim of this study. Peripheral dependent edema was observed in all CsA-treated animals. This is a common side effect in CsA-treated monkeys, and is not attributed to afucosylated antibody. Central line infection with gram negative rods caused the demise of one monkey each in the CsA monotherapy (MA029) and CsA+afucosylated antibody (MB116). This complication is observed occasionally in immunosuppressed animals with chronic central access catheters, and is not attributable to afucosylated antibody. Lymphoma was incidentally discovered at necropsy in one animal treated with CsA+afucosylated antibody (MB106). The immunochemistry of the lymphoma demonstrated a B cell phenotype with Epstein-Bar virus (EBV) antigen, demonstrating infection or reactivation by this virus. B-cell predominance and EBV detection are clinically consistent with post-transplant lymphoproliferative disease (PTLD). PTLD has occasionally been observed in immunosuppressed cynomolgus monkeys. Reactive lymphoid aggregates in the kidney in the other animal (MB116) treated with CsA+afucosylated antibody could represent an early PTLD, but EBV was not detected, morphology and immunohistochemical phenotype were indeterminate, and this diagnosis is not conclusively supported. Presence of systemic sepsis in this animal is a confounding variable that could perhaps explain the incidental finding of infiltrates in the native heart and lungs. The finding of PTLD in one animal and possibly in another, coupled with a strong trend to reduced acute rejection incidence and significantly inhibited CAV all support the conclusion that treatment with afucosylated antibodies of the invention is immunosuppressive in the context of CsA treatment.

What is claimed:

1. An antibody that has a binding affinity to CCR5 of about 10⁻¹³ to about 10⁻⁹ M (K_{D}), having a sugar chain bound thereto at Asn297, wherein the amount of fucose within said sugar chain is 65% or lower.

2. The antibody of claim 1, wherein the amount of fucose within said sugar chain is between 5% and 65%.

3. The antibody of claim 2, selected from the group consisting of the antibody wherein the amount of NGNA in said sugar chain is 1% or less, the antibody wherein the amount of N-terminal alpha-1,3-galactose in said sugar chain is 1% or less, and the antibody wherein the amount of NGNA and the amount of N-terminal alpha-1,3-galactose in said sugar chain is 1% or less simultaneously.

4. The antibody of claim 3, wherein the antibody is a chimeric, humanized, or human antibody.

5. The antibody of claim 1, wherein said antibody is selected from the group consisting of:
the antibody comprising as heavy chain complementary determining regions (CDRs) the
CDRs of SEQ ID NO: 06 and as light chain CDRs the CDRs of SEQ ID NO: 07,
the antibody comprising as heavy chain CDRs the CDRs of SEQ ID NO: 08 and as light chain CDRs the CDRs of SEQ ID NO: 09,
the antibody comprising as heavy chain CDRs the CDRs of SEQ ID NO: 10 and as light chain CDRs the CDRs of SEQ ID NO: 11,
the antibody comprising as heavy chain CDRs the CDRs of SEQ ID NO: 12 and as light chain CDRs the CDRs of SEQ ID NO: 13,
the antibody comprising as heavy chain CDRs the CDRs of SEQ ID NO: 14 and as light chain CDRs the CDRs of SEQ ID NO: 15,
the antibody comprising as heavy chain CDRs the CDRs of SEQ ID NO: 16 and as light chain CDRs the CDRs of SEQ ID NO: 19,
the antibody comprising as heavy chain CDRs the CDRs of SEQ ID NO: 16 and as light chain CDRs the CDRs of SEQ ID NO: 20,
the antibody comprising as heavy chain CDRs the CDRs of SEQ ID NO: 17 and as light chain CDRs the CDRs of SEQ ID NO: 19,
the antibody comprising as heavy chain CDRs the CDRs of SEQ ID NO: 17 and as light chain CDRs the CDRs of SEQ ID NO: 20,
the antibody comprising as heavy chain CDRs the CDRs of SEQ ID NO: 18 and as light chain CDRs the CDRs of SEQ ID NO: 19, and
the antibody comprising as heavy chain CDRs the CDRs of SEQ ID NO: 18 and as light chain CDRs the CDRs of SEQ ID NO: 20.

6. The antibody of claim 5, further characterized as showing high binding affinity to FcγRIII.

7. A pharmaceutical composition comprising an effective amount of an antibody according to claim 1 and a pharmaceutically acceptable excipient.

8. The pharmaceutical composition of claim 7, further comprising an effective amount of a a calcineurin inhibitor.

9. The pharmaceutical composition of claim 8, wherein said calcineurin inhibitor comprises cyclosporin A.

10. A method of treating or preventing acute and chronic organ transplant rejection in a subject, comprising administering to said subject an effective amount of an antibody according to claim 1.

11. The method of claim 10, further comprising administering to said subject an effective amount of a calcineurin inhibitor.

12. The method of claim 11, wherein said calcineurin inhibitor comprises cyclosporin A.

13. A CHO cell capable of recombinantly expressing GnTIII and an anti-CCR5 antibody of claim 1.

14. The CHO cell of claim 13, which further expresses recombinant ManII.
